(19) United States
(12) Patent Application Publication
(10) Pub. No.: US 2022/0392571 A1
(43) Pub. Date: Dec. 8, 2022

(54) METHOD FOR ANALYZING CELL CLUSTERS

(71) Applicant: Yeda Research and Development Co. Ltd., Rehovot (IL)

(72) Inventors: Ido AMIT, Rehovot (IL); Amos TANAY, Rehovot (IL); Amir GILADI, Rehovot (IL); Merav COHEN, Rehovot (IL)

(73) Assignee: Yeda Research and Development Co. Ltd., Rehovot (IL)

(21) Appl. No.: 17/884,594

(22) Filed: Aug. 10, 2022

Related U.S. Application Data
(63) Continuation of application No. PCT/IL2021/050161, filed on Feb. 10, 2021.

(30) Foreign Application Priority Data
Feb. 10, 2020 (IL) 272586

(51) Int. Cl. G16B 25/10

(52) U.S. Cl. CPC G16B 25/10

(57) ABSTRACT

A method of determining cell members of a cell cluster in a tissue of interest is disclosed. The cell members physically interact with one another in the cell cluster. Computer software products capable of carrying out the method are also disclosed.

### RELATED APPLICATIONS

This application is a Continuation of PCT Patent Application No. PCT/IL2021/050161, having international filing date of Feb. 10, 2021 which claims the benefit of priority of Israeli Patent Application No. 272586 filed on Feb. 10, 2020. The contents of the above applications are all incorporated by reference as if fully set forth herein in their entirety.

### FIELD AND BACKGROUND OF THE INVENTION

The present invention, in some embodiments thereof, relates to a method of analyzing cell clusters and more particularly to clusters of two or three cells.

Cellular communication is fundamental to the development and homeostasis of tissues. Tissues are organized in functional niches where cells communicate via physical interactions, exchange metabolites and ligand receptor signaling, forming cellular networks, such as neuronal synapses, lung alveoli or intestinal villi. Particularly, immune cells participate in many types of physical interactions with other immune and non-immune lineages, facilitating tissue repair, cellular education within lymph nodes, phagocytosis and cell death. Genomic technologies have revolutionized our understanding of individual cell state and its regulation, but their application in identification, quantification and characterization of cell-cell interactions is still limited.

One important example for cellular communication in the immune system is the physical interaction between T cells and antigen presenting cells. This interaction is essential for the ability of the immune system to discriminate between self and non-self. When CD4⁺ T cells physically interact with antigen-presenting cells, mainly dendritic cells (DC), they form an immune synapse involving their T cell receptor (TCR), and an antigen presented on Major Histocompatibility Complex-II (MHC-II) molecules. Depending on the affinity of the TCR to the presented antigen and the presence of costimulatory molecules, this interaction can activate intracellular signaling and gene expression, leading to specific immune decisions.

Despite being a major determinant of infectious disease and cancer outcomes, characterization of the transcriptional regulation and crosstalk of T-DC interactions in vivo remains difficult, demonstrating the challenges in capturing and defining cellular interactions at high resolution. Co-culture in vitro assays or microscopy techniques coupled to transgenic mice models with invariable TCR that recognize predesigned antigens, while effective, are limited in resolution and context. In parallel, genomic advances, and single-cell RNA sequencing (scRNA-seq) in particular, can potentially unravel the regulatory events that arise from these interactions. However, efforts to use scRNA-seq methods to study physical interactions are hampered by the destructive process of tissue dissociation, which eliminates cellular conjugates. Partial information about interactions in the tissue can be retrieved by recent breakthroughs in spatial transcriptomics and proteomics, or direct screening of ligand-receptor pairings. A different approach is to use mild dissociation that preserves cellular structures in the tissue, and focus on cell aggregates representing physically interacting cells, as was demonstrated in the bone marrow and liver lobules [Szczerba, B. M. et al. Nature 566, 553-557 (2019); Halpern, K. B. et al. Nat Biotechnol 36, 962-970 (2018); and Boisset, J. C. et al. Nat Methods 15, 547-553 (2018)].

### SUMMARY OF THE INVENTION

According to an aspect of the present invention there is provided a method of determining cell members of a cell cluster in a tissue of interest comprising:
(a) receiving a transcriptome of the cell cluster;
(b) accessing a computer readable medium storing a library having a plurality of entries, each entry having a predicted transcriptome of a cell cluster and a set of identities of known cell types forming the cell cluster;
(c) searching the library for an entry having a predicted transcriptome matching the received transcriptome; and
(d) extracting from the entry a corresponding set of identities, thereby determining the cell members of the cell cluster in a tissue, the cell members being the corresponding set of identities.

According to an aspect of the present invention there is provided a method of identifying a gene whose transcription is regulated by cell clustering comprising:
(a) obtaining data of the transcriptome of a first cell of the cluster, wherein the transcriptome is obtained when the first cell is in a single-cell state;
(b) obtaining data of the transcriptome of a second cell of the cluster, wherein the transcriptome is obtained when the second cell is in a single-cell state, wherein the first cell and the second cell express non-identical cell surface markers;
(c) obtaining a prediction of the transcriptome of a cluster of the first cell and the second cell from the data of the transcriptome of the first cell in a single-cell state and the data of the transcriptome of the second cell in a single cell state, using the null hypothesis that the transcriptome of the cluster is the combination of the transcriptome of the first cell in the single cell state and the transcriptome of the second cell in the single cell state;
(d) isolating a cluster of cells which comprise the first cell and the second cell from a heterogeneous population of cells;
(e) performing transcriptome analysis of the cluster of cells so as to obtain the true transcriptome of the cluster; and
(f) comparing the prediction to the true transcriptome, wherein expression of a gene of the true transcriptome that is statistically significantly altered is indicative of a gene that is regulated by cell clustering.

According to an aspect of the present invention there is provided a computer software product, comprising a computer-readable medium in which program instructions are stored, which instructions, when read by a data processor, cause the data processor to receive a transcriptome of a cell cluster, and to execute the method described herein.

According to an aspect of the present invention there is provided an apparatus for determining cell members of a cell cluster in a tissue of interest, the apparatus comprising a data processor configured for receiving a transcriptome of a cell cluster, and for executing the method described herein.

According to an aspect of the present invention there is provided a method of identifying cell members of a cell cluster in a tissue of interest:
(a) isolating a plurality of single cells of different cell types from the tissue of interest on the basis of expression of a unique cell marker;
(b) determining the transcriptomes of the single cells of different cell types;
(c) obtaining predictions of the transcriptomes of a plurality of clusters of non-identical cells of the plurality of single cells from the transcriptomes of the single cells, each of the plurality of clusters comprising a unique combination of cells, wherein the predictions are based on the null hypothesis that the transcriptome of a cluster is the sum of a transcriptome of the individual cells of the cluster when in a single cell state;
(d) isolating a cluster of cells from the tissue;
(e) performing transcriptome analysis of the cluster of cells so as to obtain the true transcriptome of the cluster;
(f) comparing the predictions to the true transcriptome; and
(g) identifying the members of the cell cluster by selecting the transcriptome prediction that has the closest identity to the true transcriptome.

According to an aspect of the present invention there is provided a method of ascertaining a status of a tissue comprising identifying cell members of cell clusters of the tissue as described herein, wherein a presence or an increase in a number of cell clusters comprising a combination of cell members indicative of a tissue status, is indicative of a status of the tissue.

According to embodiments of the present invention, the known cell types comprise at least 5 different cell types.

According to embodiments of the present invention, each of the cell types comprises a unique surface marker or a unique combination of cell surface markers.

According to embodiments of the present invention, the tissue is not hepatic tissue.

According to embodiments of the present invention, the cell cluster consists of two cells.

According to embodiments of the present invention, the cell cluster consists of a dendritic cell and a T cell.

According to embodiments of the present invention, the cell cluster consists of three cells.

According to embodiments of the present invention, the tissue is selected from the group consisting of lymph node, bone marrow and lung tissue.

According to embodiments of the present invention, the known cell types comprises a plurality of cell states of an identical cell.

According to embodiments of the present invention, the different cell types comprises at least 5 different cell types.

According to embodiments of the present invention, the unique cell marker comprises a unique combination of cell markers.

According to embodiments of the present invention, the unique cell marker comprises a cell surface marker.

According to embodiments of the present invention, the isolating a plurality of single cells is effected by flow cytometry or using a microfluidics device.

According to embodiments of the present invention, the isolating the cluster of cells is effected by flow cytometry or using a microfluidics device.

According to embodiments of the present invention, the tissue is not hepatic tissue.

According to embodiments of the present invention, the cluster consists of two cells.

According to embodiments of the present invention, the cluster consists of a dendritic cell and a T cell.

According to embodiments of the present invention, the cluster consists of three cells.

According to embodiments of the present invention, the tissue is selected from the group consisting of lymph node, bone marrow and lung cells.

According to embodiments of the present invention, the different cell types comprise different cell states.

According to embodiments of the present invention, the method further comprises identifying genes of the cells whose transcription is regulated by cell clustering following step (g).

According to embodiments of the present invention, the method further comprises determining the abundance of a cell cluster of a particular combination.

According to embodiments of the present invention, the status of a tissue comprises a disease.

According to embodiments of the present invention, the disease is selected from the group consisting of cancer, an infectious disease, fibrosis and an immune disease.

According to embodiments of the present invention, the immune disease is an autoimmune disease.

According to embodiments of the present invention, the heterogeneous population of cells comprises at least 5 cell types.

According to embodiments of the present invention, the cluster does not comprise hepatocytes.

According to embodiments of the present invention, the cluster consists of two cells or three cells.

According to embodiments of the present invention, the method further comprises isolating the first cell and the second cell from the heterogeneous population of cells such that they are in a single cell state prior to step (a).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or apparatus of embodiments of the invention can involve performing or completing selected tasks manually, automatically, or a combination thereof. Moreover, according to actual instrumentation and equipment of embodiments of the method and/or apparatus of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or apparatus as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-H—detection and sequencing of physically interacting cells. A, Schematics: using PIC-seq to characterize interactions between T cells (green) and dendritic cells (DC; red). B, Representative FACS plot of co-cultured CD4⁺ T cells and CD11c⁺ DC. Gates demarcating single and double positive populations are shown. PIC (black gate) are double positive for TCRβ and CD11c epitopes; n=3 independent experiments. Population frequencies represent mean±SEM. C, A 2-dimensional projection of 3,650 TCRβ⁺T cells and 3,856 CD11c⁺ DC, grouped into 127 metacells, generated by the MetaCell algorithm. Dots represent single cells and dot colors relate to cell subsets. D, Gene expression profiles of 7,506 single cells grouped into 9 transcriptional subsets. Top panels indicate unique molecular identifier (UMI) count, time point of collection (3, 20 or 44 h), and whether a cell is derived from mono- or co-culture. E, DC and T cell subset identity of single cells in D. F, Gene expression profiles of 2,726 PIC, grouped by their contributing T cell and DC identities, as determined by PIC-seq algorithm. Top panels as in D. G, DC and T cell subset identity of PIC contributing cells in F, as determined by PIC-seq algorithm. H, Estimation of the relative UMI count from T cells (green), and DC (red) contributing to each PIC in E, as inferred by PIC-seq algorithm (mixing factor).

FIGS. 2A-D—T cells interacting with PIC show early activation and differentiation. A-B, Distribution of T (A) and DC (B) subsets along the three time points when grown in mono-culture, co-culture or as contributing cells to PIC. Two-tailed Fisher's exact test; *p<0.05 C, Observed gene-expression levels in PIC plotted against their expected levels as determined by PIC-seq, pooled over all PIC. Genes with observed/expected ratio>2 are highlighted, and colored by their specificity in the T (green) or DC (red) expected contributions (log 2 fold change between the two background populations). D, Mean observed and expected gene-expression levels in PIC grouped according to their T and DC contributor identities (as in FIG. 1G) along the different time points of the co-culture experiment. Expected values are decomposed by the contribution from the T (green) and DC (red) PIC components. Bottom panel shows T and DC subset identities. Dashed box highlights PIC of proliferating T cells. Error bars indicate binomial 95% confidence intervals of the observed values.

FIGS. 3A-L—Studying T-myeloid interactions in an in vivo infection model. A, Confocal microscopy image of a LN 48 hours after infection with fluorescently labelled Nb. Arrows point to rare interactions between TCRβ⁺ T cells and a CD11c⁺ Nb. antigen-presenting myeloid cells. Scale bar 30 μm. B, Comparison between percentages of non-conjugated T cells and DC, and T-DC PIC achieved by mild and strong dissociation conditions with the enzymes liberase and DNaseI. C, Experimental design. Mice were injected intra-dermally into the ear pinna with fluorescently labelled Nb or PBS control. Draining auricular LN were extracted 48 h following infection, and prepared for PIC-seq. D, Representative FACS plot of T cells (green gate), DC (red gate) and PIC (black gate) purified from auricular LN; n=6 independent experiments. Population frequencies represent mean±SEM. E, Confocal microscopy images of TCRβ⁺CD11c⁺PIC. Scale bar=10 μm. F, Quantification of doublets and triplets in TCRβ⁺CD11c⁺PIC derived from naïve auricular LN. LN from two ears were pooled from each sample; n=4 biological replicates; Values represent mean±SEM. G, A 2-dimensional projection of 4,313 TCRβ⁺T and 3,149 CD11c⁺ APC, grouped into 71 metacells, generated by the MetaCell algorithm. Dots represent single cells, and dot colors and labels relate to annotation of T and APC subsets. H, Gene expression profiles of 7,462 single cells grouped into 10 cell types. Top panel indicates UMI count, whether a cell is from NB-injected or PBS control LN, and whether a cell is labelled by Nb-derived antigen. I, APC and T cell subset identity of single cells in H. J, Gene expression profiles of 2,667 PIC, grouped by their contributing T and APC identities, as determined by PIC-seq algorithm. Top panels as in H. K, APC and T cell subset identity of PIC contributing cells in J, as determined by PIC-seq algorithm. L, Estimation of the relative UMI count from T cells (green), and APC (red) contributing to each PIC in J, as inferred by PIC-seq algorithm (mixing factor).

FIGS. 4A-I—Regulatory T cells have high capacity to interact with APC. A-B, Differences in metacell composition between (a) PIC contributing T cells and (B) PIC contributing APC in PBS-injected control LN. Each bar represents one metacell, and shows log 2 fold change of its frequency between the PIC and the TCRβ⁺ (A) or CD11c⁺ (B) single-cell populations. Bar colors relate to metacell subset identity. C-D, same as (A-B) for Nb-infected LN. E, Representative FACS plot of Foxp3 expression levels in the TCRβ⁺ non-conjugated T cells, and PIC-derived T cells. Population frequencies represent mean±SEM; Results are representative of two independent experiments. F, Representative image of confocal microscopy of Foxp3⁺TCRβ⁺ CD11c⁺ PIC isolated from naïve auricular LN. Scale bar=15 μm. G, Mean observed (gray bar) and expected (colored bar) gene expression in Treg-myeloid PIC in different subsets of myeloid cells (x-axis). Expected values are decomposed by their contribution from the T (green) and DC (red) PIC components. Error bars indicate binomial 95% confidence intervals of the observed values. H, Mean observed (gray bar) and expected (colored bar) gene expression in PIC of migratory DC and T cell. Shown are PIC from PBS control, Nb-injected mice (Ag⁻), and Nb-presenting (Ag⁺) PIC. Expected values are decomposed by their contribution from the T (green) and DC (red) PIC components. Error bars indicate binomial 95% confidence intervals of the observed values. I, Mean fluorescence intensity (MFI) of the protein DLL4 expressed by pathogen presenting and non-presenting CD11c⁺ APC and PIC. n=2 biological replicates.

FIGS. 5A-H: PIC-seq performance in a co-culture experiment. A, Spurious PIC rate is quantified by mixing two parallel cultures, one stained for TCRb-FITC and CD11c-APC, and the other to TCRb-PE and CD11c-APC.Cy7. PIC from FITC+APC.Cy7+ and PE+APC+ populations are considered spurious (˜1% of all PIC). B, PIC share genes from T and DC. Shown is expression of top 10 differential DC genes (y axis) plotted against top 10 differential T genes (x axis) in single cells (left) and in PIC (right). C, Performance of the linear regression model, used to estimate the mixing factor of 20,000 synthetic PIC. D-E, Performance of the (d) T cell and (e) DC metacell assignments of PIC-seq over 5,000 synthetic PIC. Each row summarizes all synthetic PICs originating from one metacell and their assignments to metacells by PIC-seq (columns. Data is row-normalized). F, Estimation of triplet frequency in PIC-seq data. Triplets can be composed of two T cells and a DC (left), or of one T cell and two DC (right). Each PIC was assigned a triplet score, indicating the improvement in likelihood when instead of modelling doublets, the MLE models heterotypic triplets. Plotted are the cumulative distributions of triplet score of synthetic doublets (gray), triplets (blue), and empirical PIC (green). G, Pearson correlation between genes' observed and expected values across all empirical PICs, in a leave 10% out cross-validation. Expected values are calculated (i) from the full PIC-seq algorithm, (ii) when incorporating the meta-cell assignments or (iii) the mixing factor only, or (iv) when mixing factor and metacell assignments are arbitrary. H, Differential gene expression between singlets from three DC subsets against all other DC (log 2 fold change, x axis), plotted against differential expression between PIC assigned to the same DC subsets and the activated T cell subset, calculated against all other PIC assigned to activated T cells (log 2 fold change, y axis).

FIGS. 6A-D: PIC-seq reveals advanced differentiation in conjugated T cells. A, Schematics: expected PIC gene expression values are calculated by mixing the multinomial probability vectors of the contributing metacells by the inferred mixing factor, while preserving total UMI count. B, Differences between observed and expected gene expression pooled over all PIC (log 2 fold change). Expected values are calculated over the maximum likelihood doublet assignments (x axis), or the maximum likelihood triplet assignments (y axis; of two T and one DC; Methods). C, Mean observed and expected gene-expression levels in PIC grouped by their T and DC contributor subsets along the different time points of the co-culture experiment, shown for selected genes. Expected values are decomposed by the contribution from the T (green) and DC (red) PIC components. Bottom panel shows T and DC subset identities. Error bars indicate binomial 95% confidence intervals of the observed values. D, Log2 fold change values of 348 genes exhibiting significant differences between their observed and expected values across PIC grouped by their T and DC contributor subsets. c2 test; q<10⁻⁶.

FIGS. 7A-J: PIC-seq in the postnatal lung. A, FACS plot of EPCAM+ (CD326) epithelial cells, CD45+ immune cells and PIC derived from postnatal murine lung. B, Gene expression profiles of 1,071 CD45+ or EPCAM+ single cells from postnatal lungs, grouped into 13 cell types. Top panel indicates UMI count. C, CD45+ and EPCAM+ cell subset identity of single cells in A. D, Gene expression profiles of 543 PIC, grouped by their contributing CD45+ and EPCAM+ identities, as determined by PIC-seq algorithm. Top panels as in A. E, CD45+ and EPCAM+ subset identity of PIC contributing cells in b, as determined by PIC-seq algorithm. F, Estimation of the relative UMI count from epithelial cells (green), and immune cells (red) contributing to each PIC in B, as inferred by PIC-seq algorithm (mixing factor). G-H, Differences in metacell composition between PIC contributing epithelial cells (G) and PIC contributing immune cells (H) in postnatal lungs. Each bar represents one metacell, and shows log2 fold change of its frequency between the PIC and the EPCAM+ (G) or CD45+ (H) single-cell populations. Bar colors relate to metacell subset identity. I, Distribution of Epcam+ and CD45+ subsets in non-conjugated populations and in PIC. Two-tailed FDR adjusted Fisher's exact test; *q<0.05; **q<0.001; ***q<10-5. J, Mean observed (gray bar) and expected (colored bar) gene expression of Alveolar type 1 (AT1)-C45+ PIC grouped by subsets of C45+contributing cells (x-axis). Error bars indicate binomial 95% confidence intervals of the observed values.

FIGS. 8A-F: PIC-seq application on tumor and adjacent normal tissues derived from stage-I biopsies of NSCLC patients. (A) Schematics of the experimental paradigm using PIC-seq to characterize interactions between myeloid and T cells in human clinical specimens. (B) Representative FACS plots of CD64⁺CD11c⁺ myeloid (purple) and TCRβ⁺ T (blue) singlets, and CD64⁺CD11c⁺TCRβ⁺ PICs (orange) purified from normal lung and TME of NSCLC patients (n=8 independent experiments); Population frequencies represent mean±s.e.m. (C). A two-dimensional (2D) representation of a Metacell model of 3,688 TCRβ⁺ T cells and 4,529 CD11c⁺CD64⁺ myeloid cells, grouped into 17 T and myeloid subsets. Dots represent single cells, and dot colors are related to annotation of T and myeloid cell subsets. (D) Projection of TCRβ⁺ T or CD64⁺CD11c⁺ myeloid sorted cells derived from normal lung tissue or TME onto the 2D map represented in C. (E) A pairwise gene correlation analysis of the representation of T and myeloid subsets in samples derived from TME and adjacent normal tissues of NSCLC patients. (F) Enrichment of T and myeloid subsets in the TME compared to the matched adjacent normal lung tissue across the eight NSCLC profiled patients. Each circle represents one patient. TME—Tumor microenvironment.

FIGS. 9A-F: Interaction preferences of T and myeloid subsets revealed by PIC-seq. (A) Distribution of T subsets in TCRβ+ singlet T and CD64⁺CD11c⁺TCRβ⁺ PIC in normal lung tissue and the TME of NSCLC patients. Colors are related to annotation of T cell subsets. Cells are pooled from all profiled patients. FDR adjusted two-tailed Fisher's exact test. (B) Gene expression profiles of singlet T cells (left) and PICs (right) grouped by their metacell and PIC-seq assignment to T subsets. Shown are genes supporting assignment of singlets and PICs to the CD4⁺PD-1⁺CXCL13⁺ T subset. Bottom panels indicate metacell and PIC-seq assignment of T identities. (C) Comparison of different T subset frequencies in singlet and PICs derived from normal tissue and TME across all profiled patients. Mann-Whitney test. (D) Distribution of myeloid subsets in CD64⁺CD11c⁺ singlet myeloid and CD64⁺CD11c⁺TCRβ⁺ PICs in normal tissue and TME. Colors are related to annotation of myeloid cell subsets. Cells are pooled from all profiled patients. FDR adjusted two-tailed Fisher's exact test. (E) Gene expression profiles of singlet myeloid cells (left) and PICs (right) grouped in to their metacell and PIC-seq assignment to myeloid subsets. Shown are genes supporting assignment of singlets and PICs to the mregDC subset. Bottom panels indicate metacell and PIC-seq assignment of myeloid identities. (F) Comparison of different myeloid subset frequencies in singlet and PICs derived from normal and tumor tissues across all profiled patients. Mann-Whitney test. *P<0.05, **P<0.001, ***P<10⁻⁵.

FIG. 10 is a diagram of a representative example of a library which can be stored on computer readable medium according to some embodiments of the present invention.

### DESCRIPTION OF SPECIFIC EMBODIMENTS OF THE INVENTION

The present invention, in some embodiments thereof, relates to a method of analyzing cell clusters and more particularly to clusters of two or three cells. Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Whole tissue single cell RNA sequencing (scRNA-seq) methods are now widely used to compile detailed maps of cellular states in mammalian tissues. However, current single cell technologies profile individual cells with limited spatial information, thus losing the in situ cellular context. The existence of cell-aggregates in single cell suspension in the form of doublets has been recognized as a source of contaminations in FACS and scRNA-seq analysis. As a result, several methods are now available to detect and eliminate doublets from scRNA-seq analysis. Furthermore, recent publications successfully profiled cell doublets (Szczerba, B. M. et al. Nature 566, 553-557 (2019); Halpern, K. B. et al. Nat Biotechnol 36, 962-970 (2018); Boisset, J. C. et al. Nat Methods 15, 547-553 (2018)), showing the potential impact of technologies that target and characterize physical interactions.

The present inventors now introduce PIC-seq, a technology based on doublet enrichment, capture and sequencing with improved experimental and analytical design. PIC-seq is able to capture and molecularly analyze crosstalk between cells, a process with high impact on cell signaling and differentiation. By using an experimental design to preserve, identify, enrich and collect physically interacting cells (PIC), as well as a computational framework that allows deconvolution of each PIC particle into its contributing cell types, the present inventors show PIC-seq to be applicable for inference of in situ cellular interactions and characterization of their molecular crosstalk.

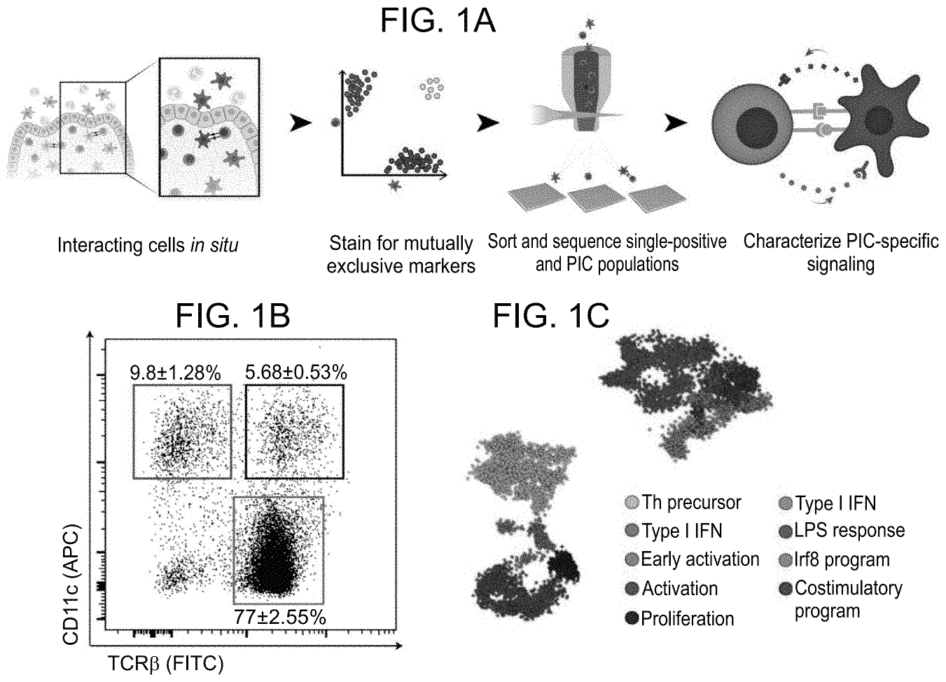

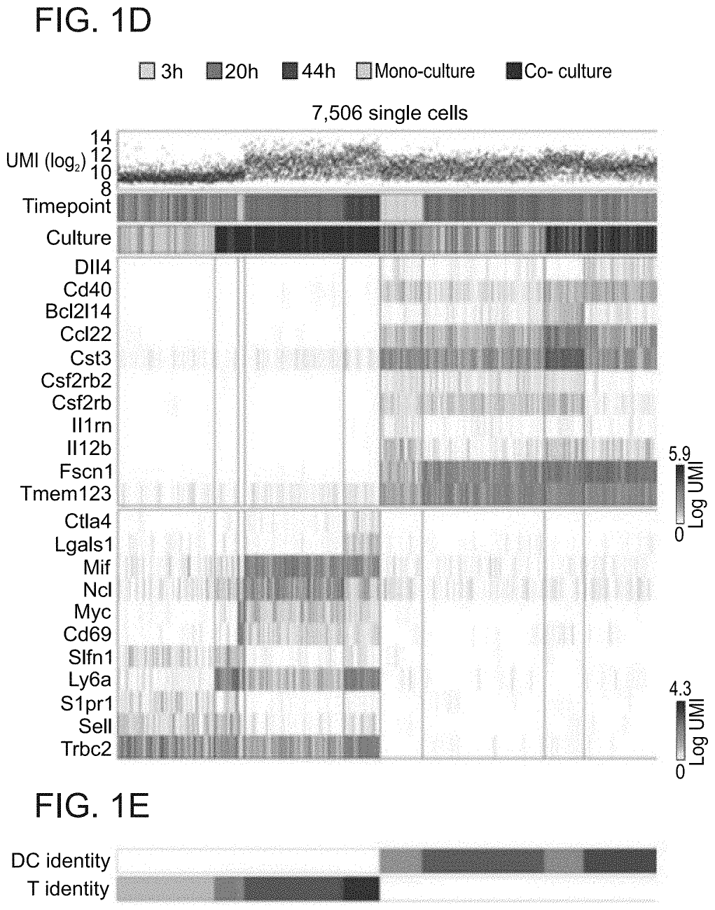

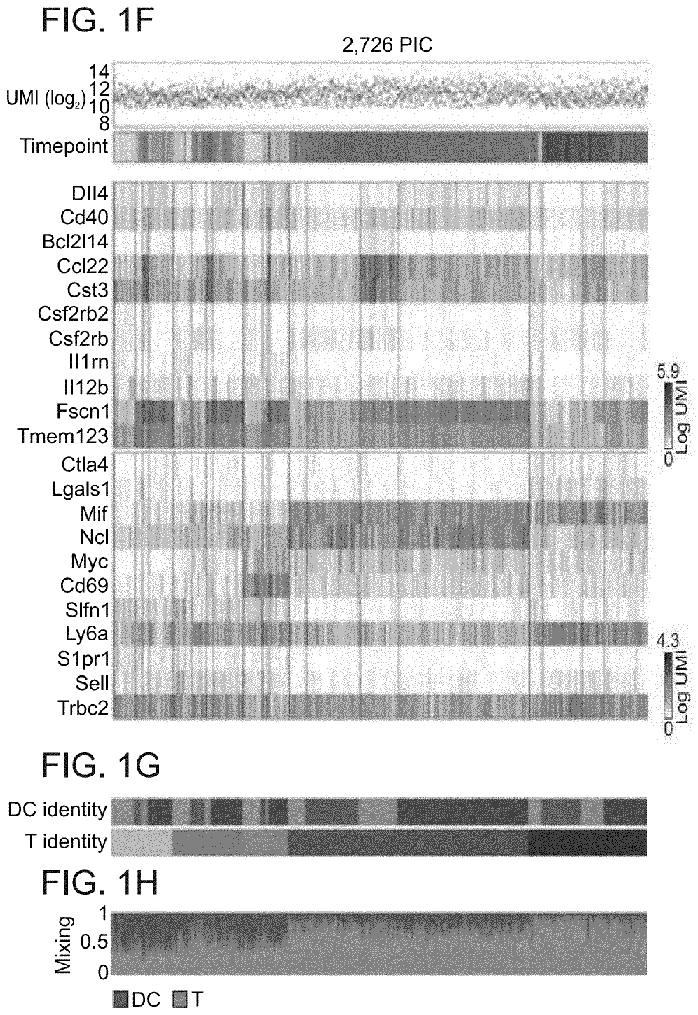

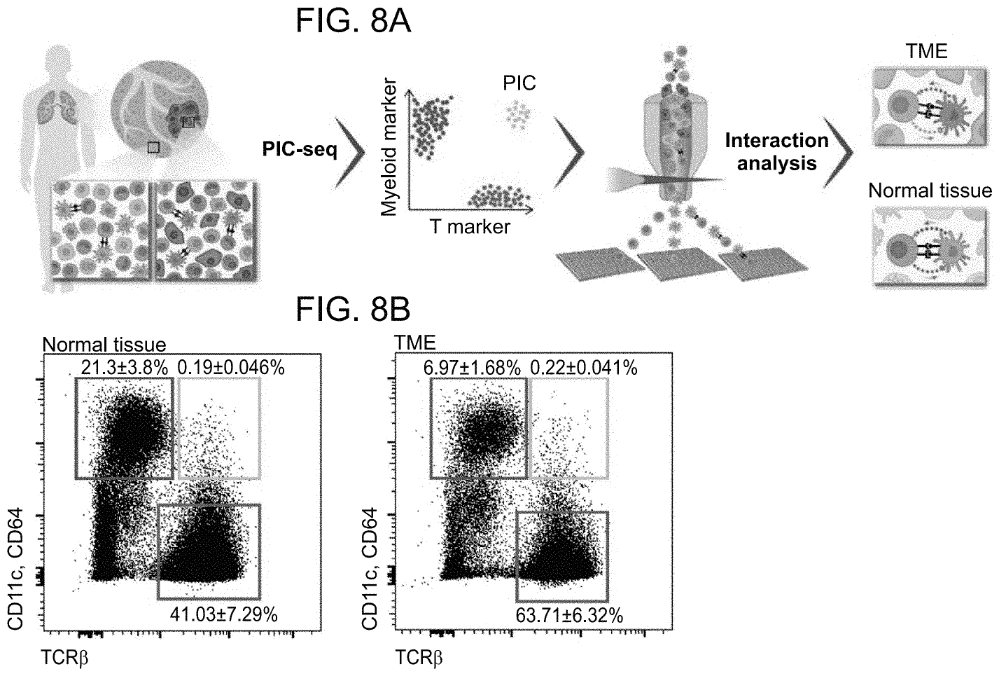

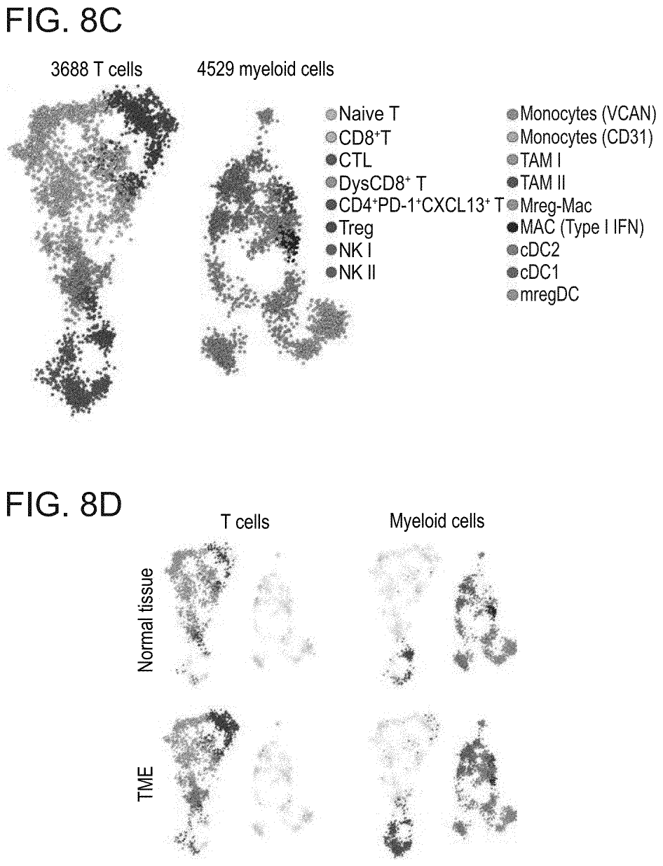

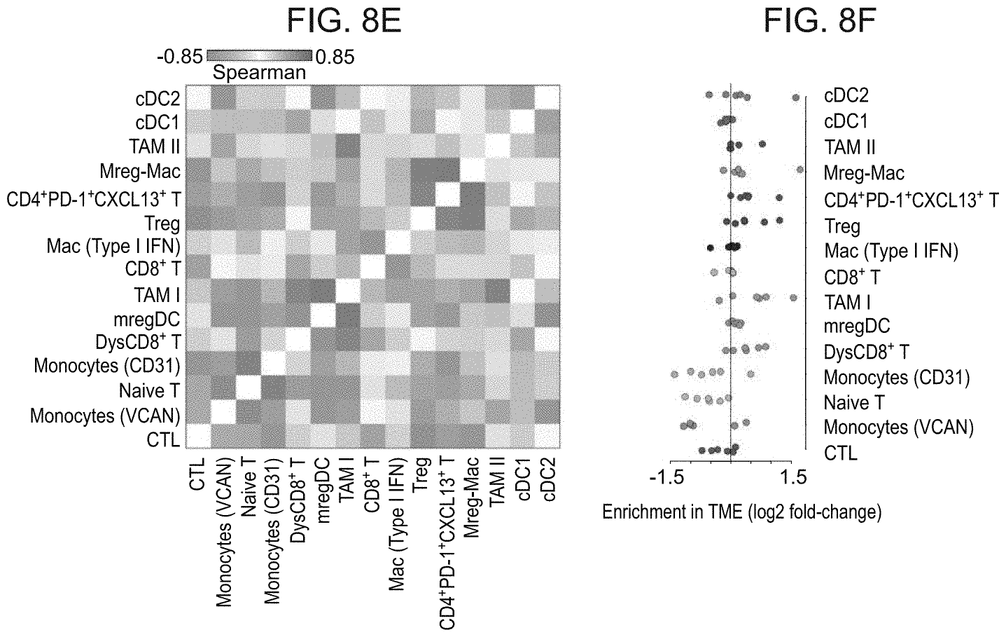

When performing a PIC-seq experiment, cells are stained for two mutually-exclusive fluorescent markers, dividing all desired cells into three populations: two “singlet” populations, each composed of a distinct group of cell types, and a double-positive PIC population which represents physical conjugates composed of cells from both singlet populations (see FIGS. 1A and 8A).

The level of contamination in the PIC population caused by spurious doublet aggregates formed during sample preparation is quantified via mixing two parallel experiments from different biological replicates, performing sample preparation in one tube, and assessing numbers of cross-biological replicate events. Using flow cytometry, cells from the two singlet populations and from the PIC population are sorted into 384 plates, lyzed and processed for single cell RNA library preparation and sequencing (MARS-seq).

The PIC-seq algorithm uses the clustering model obtained from the singlet populations to perform in silico simulation of doublets, followed by deconvolution of each sorted PIC by inferring the transcriptional identity of its contributing cells.

The PIC-seq algorithm is then able to reconstruct the expected gene expression profile of each PIC, according to the null model—assuming that the interaction itself did not initiate de novo transcription. Comparing the sorted PIC's observed to the expected transcription, and testing for differential gene expression can then identify novel transcripts unique to the PIC and absent in any of the contributing single cells.

PIC-seq can also test for the existence of triplets in the PIC population, estimate the number of triplets, filter them out or control for their existence.

The PIC-seq algorithm can be extended to detect conjugates of physical interacting cells in publicly available single cell RNA-seq datasets or data sets generated on microfluidic droplets devices.

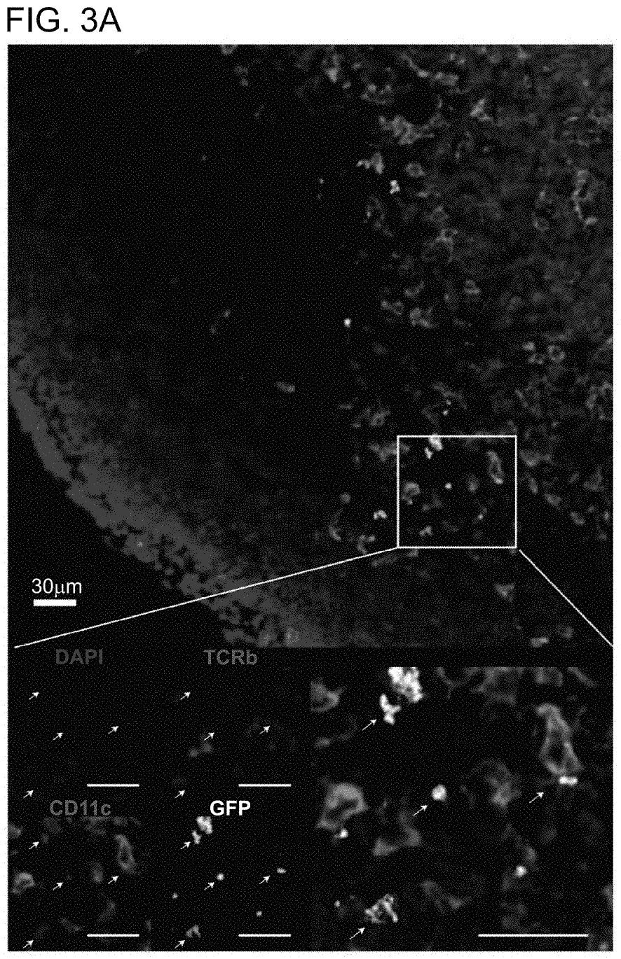

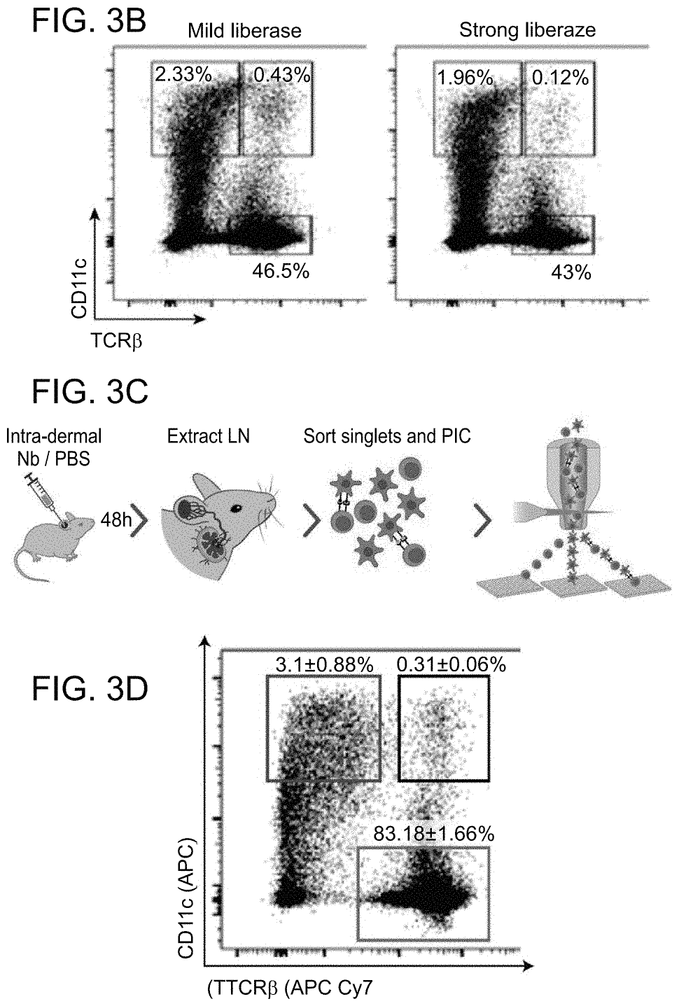

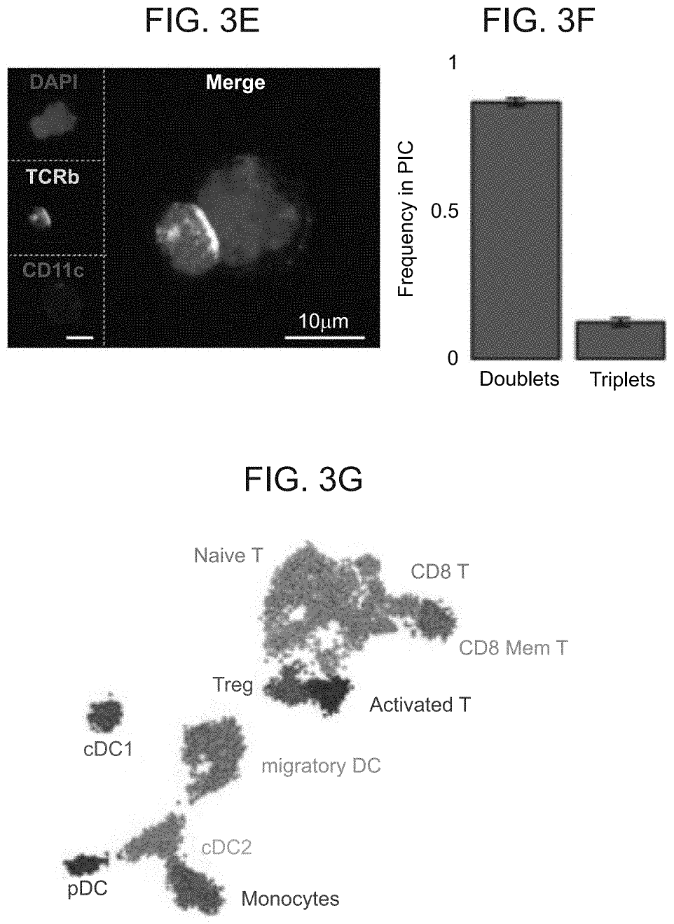

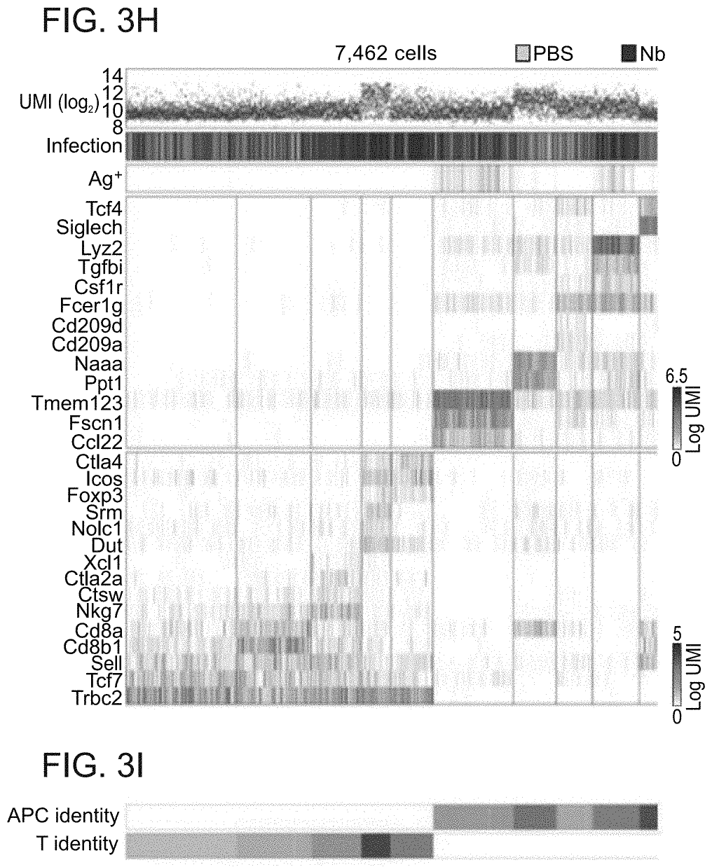

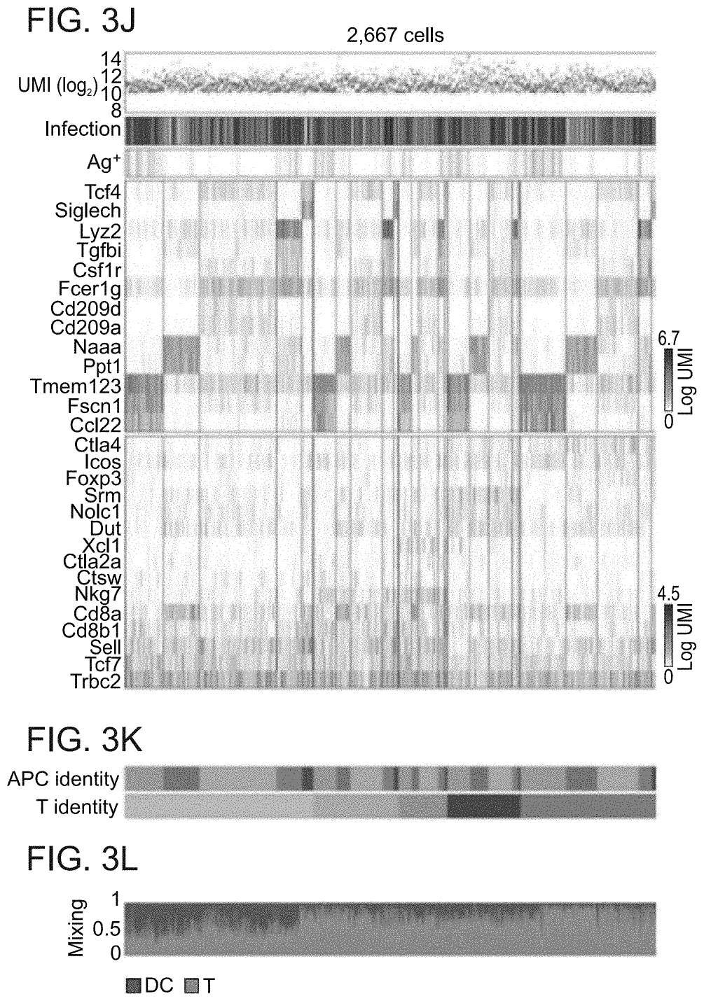

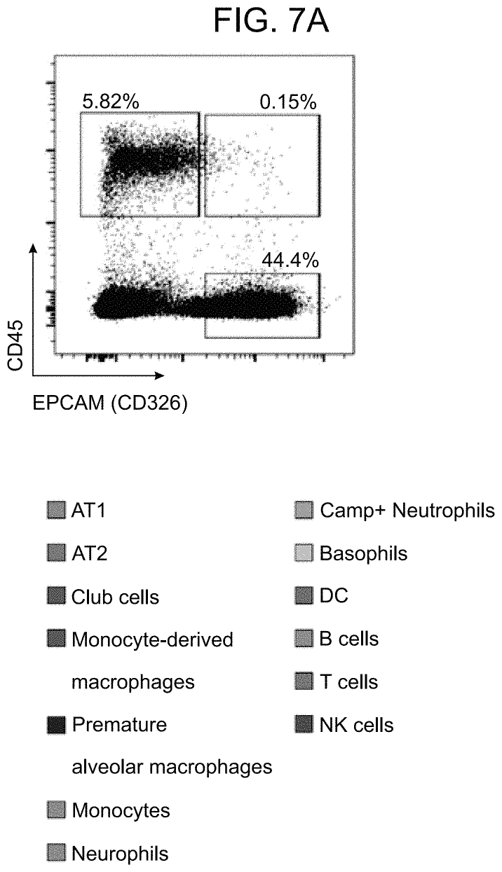

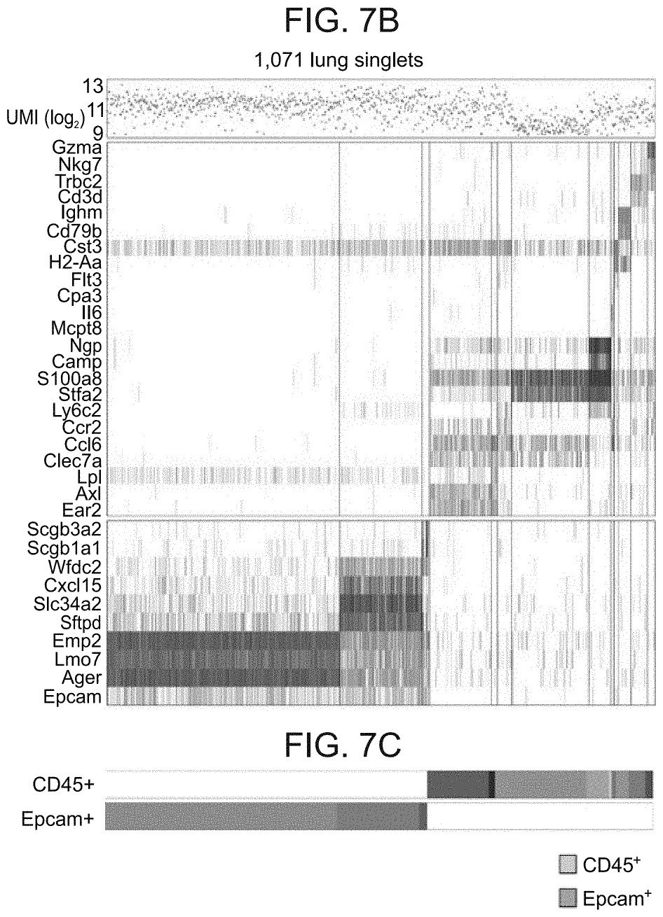

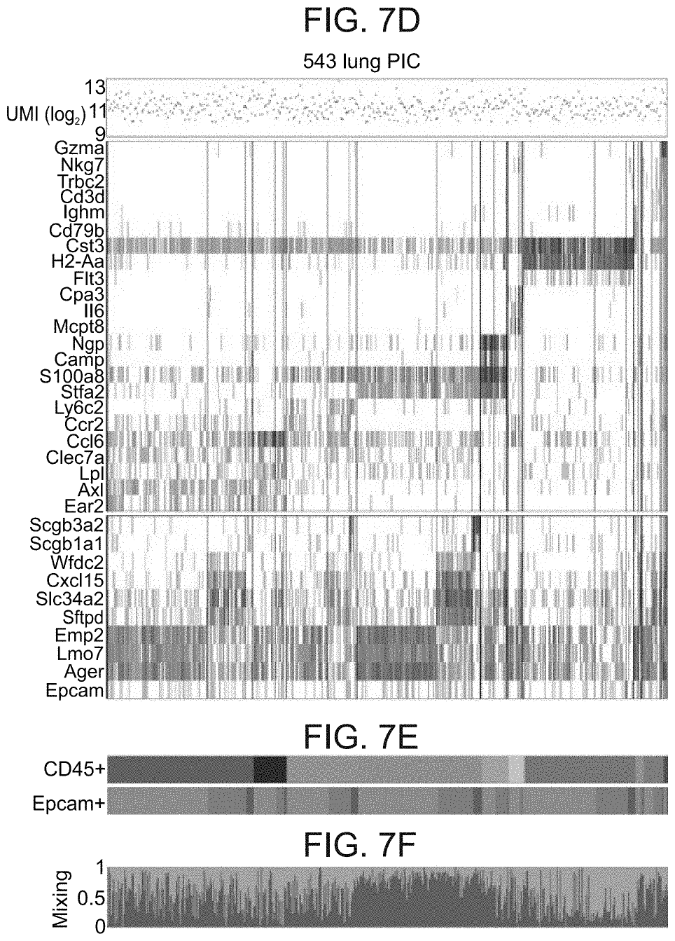

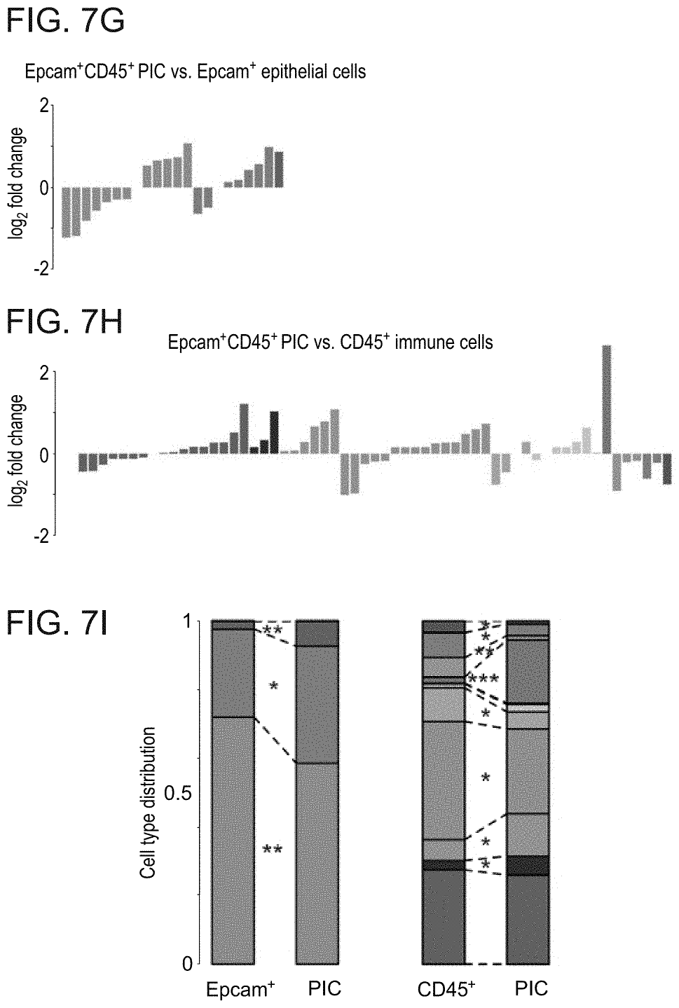

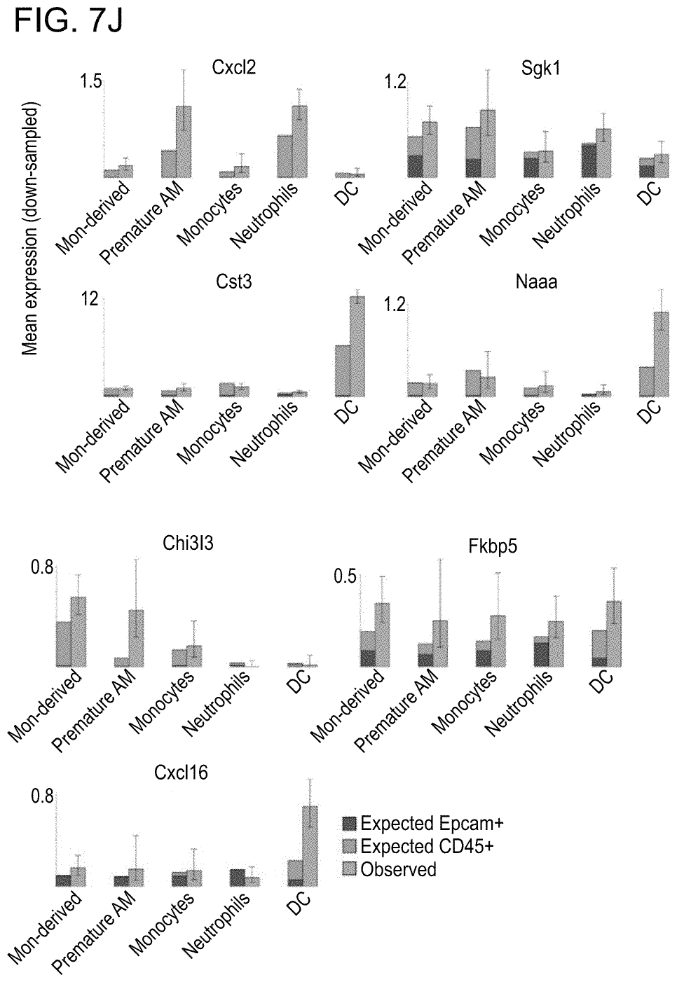

The present inventors exemplified three scenarios in which the technology can be applied: first by focusing on interactions between two specific cell type (e.g. T-DC) discriminated by cell surface markers (TCRβ and CD11c) as shown in FIGS. 3A-L and 4A-I, second by capturing cell-cell interactions between broader lineages (immune-epithelial), using pan-lineage surface markers (CD45 and EPCAM) as shown in FIGS. 7A-J; and third by focusing on interactions between myeloid and T cells in the tumor microenvironment (FIGS. 8A-F).

Importantly, by using transgenic mice, adoptive transfer or fluorescent dyes, PIC-seq can be expanded to profile interactions between populations that cannot be delineated by cell surface markers. This can open many new venues for investigation of cell-cell interactions, and profiling of PIC composed of tumors, stromal, tissue-resident and immune cells. In theory, PIC-seq can also be adapted for profiling higher orders of interactions, such as cell triplets. Importantly, PIC-seq can be applied on human samples, where invasive and genetic tools are inherently limited.

According to a first aspect of the present invention there is provided a method of determining cell members of a cell cluster in a tissue of interest comprising:
(a) receiving a transcriptome of the cell cluster;
(b) accessing a computer readable medium storing a library having a plurality of entries, each entry having a predicted transcriptome of a cell cluster and a set of identities of known cell types forming said cell cluster;
(c) searching said library for an entry having a predicted transcriptome matching said received transcriptome; and
(d) extracting from said entry a corresponding set of identities, thereby determining the cell members of the cell cluster in a tissue, the cell members being said corresponding set of identities.

Any of the methods described herein can be embodied in many forms. For example, it can be embodied in on a tangible medium such as a computer for performing the method operations. It can be embodied on a computer readable medium, comprising computer readable instructions for carrying out the method operations. It can also be embodied in electronic device having digital computer capabilities arranged to run the computer program on the tangible medium or execute the instruction on a computer readable medium.

Computer programs implementing the method of the present embodiments can commonly be distributed to users on a distribution medium such as, but not limited to, CD-ROMs or flash memory media. From the distribution medium, the computer programs can be copied to a hard disk or a similar intermediate storage medium. In some embodiments of the present invention, computer programs implementing the method of the present embodiments can be distributed to users by allowing the user to download the programs from a remote location, via a communication network, e.g., the internet. The computer programs can be run by loading the computer instructions either from their distribution medium or their intermediate storage medium into the execution memory of the computer, configuring the computer to act in accordance with the method of this invention. All these operations are well-known to those skilled in the art of computer systems.

The computational operations of the method of the present embodiments can be executed by a computer, either remote from the subject or near the subject. When the computer is remote from the subject, it can receive the data over a network, such as a telephone network or the Internet. To this end, a local computer can be used to transmit the data to the remote computer. This configuration allows performing the analysis while the subject is at a different location (e.g., at home), and also allows performing simultaneous analyses for multiple subjects in multiple different locations.

The computational operations of the method can also be executed by a cloud computing resource of a cloud computing facility. The cloud computing resource can include a computing server and optionally also a storage server, and can be operated by a cloud computing client as known in the art.

As used herein, the phrase “cell cluster” refers to a group of at least two different cells of a tissue that are in physical contact with one another.

The cell clusters typically comprise 2 or 3 cells. Preferably the cells of the clusters are non-identical—i.e. are of a different type or of the same type, but in a different state (e.g. activated s. non-activated). Preferably the cells of the clusters can be unambiguously separated by cell markers e.g. cell surface markers.

In one embodiment, the physical interaction between the cells of the cluster is a receptor ligand interaction.

In one embodiment, the cells in the cluster are viable cells.

The cells may be derived from any animal—e.g. human, rodent (e.g. mouse), sheep, monkey etc.

The cells may be derived from an animal that serves as a model for a human disease.

According to another embodiment, the physical interaction between the cells of the cluster is a tight junction (e.g. via one of the following transmembrane proteins are occludin, claudin, junctional adhesion molecules (JAMs) and tricellulins).

According to still another embodiment, the physical interaction between the cells of the cluster is via an adherens junction and/or desmosomes.

According to a particular embodiment, at least one of the cells in the cluster is an immune cell—e.g. lymphocyte, B cell, T cell, phagocytic cell, granulocyte and dendritic cell, natural killer cell etc.

In another embodiment, at least one of the cells is a myeloid cell.

Particular clusters contemplated by the present inventors are exemplified in the Examples section herein below (e.g. dendritic cell and a T cell).

According to a particular embodiment, at least one of the cells in the cluster is a cancerous cell.

In one embodiment, the cluster does not comprise neuronal cells.

Tissue samples may be obtained by any medical procedure (e.g. biopsy procedures) as is known in the art.

The tissue of interest can comprise 5 different cell types, 6 different cell types, 7 different cell types, 8 different cell types, 9 different cell types, 10 different cell types, 15 different cell types, 20 different cell types, or more.

Exemplary tissues of interest include, but are not limited to lymph node, bone marrow, cardiac tissue, skin tissue, pancreatic tissue and lung tissue. In one embodiment the cell cluster is not derived from hepatic tissue.

According to a particular embodiment, the cells are derived from a transgenic animal wherein the animal expresses a cell-specific protein that is modified to comprise a detectable moiety (e.g. a fluorescent moiety, as further described herein below).

According to another embodiment, the cells of the cell cluster are originally derived from different sources, and prior to generating the cluster, the cells in their original source are labeled such that they can be distinguished from one another—e.g. using fluorescent dyes—for example cells from peripheral blood are stained while in circulation, and at a later time point clusters of cells composed of tissue cells (e.g. cancer, epithelium etc.) and previously-stained migrating cells are isolated and analyzed—see for example Chrisopher Parish., Immunology and Cell Biology (1999) 77, 499-508. Alternatively, one of the cell types is part of a cell transplant and were labeled prior to transplantation.

In order to obtain cell clusters, the tissue (or part thereof) is subjected to a dispersing agent such that cell clusters of 2 or 3 cells are obtained.

Cell clusters are isolated using methods known in the art. The particular method is selected according to the tissue being analyzed. In one embodiment, the cell clusters are isolated using a flow cytometer.

Protocols for dispersing cells are known in the art and include for example use of enzymes such as liberase (see for example Cell Syst. 2019 Feb. 27;8(2):109-121.e6. doi: 10.1016/j.cels.2019.01.001); collagenase type IV (see for example Cell. 2017 May 4;169(4):750-765.e17. doi: 10.1016/j.cell.2017.04.014); elastase (see for example Nature. 2014 May 15;509(7500):371-5. doi: 10.1038/nature13173. Epub 2014 Apr. 13); Miltenyi lung dissociation kit (see for example: Cell. 2018 Nov. 1;175(4):1031-1044.e18. doi: 10.1016/j.cell.2018.09.009. Epub 2018 Oct. 11) and ecutase (see for example J Vis Exp. 2015 May 21;(99):e52863. doi: 10.3791/52863.

One of skill in the art would be able to calibrate dispersion protocols such that clusters of mostly two or three cells are obtained by modifying the enzyme concentration, incubation time, and/or processing (e.g. amount of chopping, amount of mechanical dissociation, amount of vortexing, amount of manual pipetation).

Once a mixture of cell clusters are generated, they may be isolated from one another according to a particular phenotype.

In one embodiment, the clusters are isolated according to expression of at least two cell-specific molecules, the first being specific to the first cell type of the cell cluster and the second being specific to the second cell type of the cell cluster.

The cell-specific molecule may be a lineage or pan-lineage marker (e.g. CD45, EPCAM, TCRβ or CD11c) or the cell-specific molecule may be a molecule that is unique to one particular cell type.

The cell specific molecule may be one that can be detected directly (e.g. a fluorescently labeled protein from a transgenic mouse, e.g. GFP fused to a lineage-specific intracellular protein). Alternatively, the cell specific molecule may be one that binds to a second molecule, the second molecule being one that can be detected directly.

In one embodiment, the cell-specific molecule is a protein (e.g. a cell surface protein or an intracellular protein). In this case, the second molecule can be an antibody which comprises a detectable moiety.

In another embodiment, the cell-specific molecule is a nucleic acid. Methods of labeling nucleic acids are known in the art and include the use of nucleic acid probes which are attached to a detectable moiety.

The detectable moiety may be a fluorescent moiety, a phosphorescent moiety, a radioactive moiety or a color moiety (such as emitted by a chromophore).

In one embodiment the detectable moiety is a fluorescent moiety.

Fluorescent moiety: Fluorescent moieties (also referred to as fluorophores) are chemical compounds that absorbs light energy at one wavelength and nearly instantaneously emits light at another, longer wavelength of lower energy. The fluorescent moiety of the oligonucleotide probes of the present invention may be compounds that produce chemiluminescence when excited by chemical reaction. Most fluorescent moieties are either heterolytic or polyaromatic hydrocarbons. The fluorescence signature of each individual fluorescent moiety is unique in that it provides the wavelengths and amount of light absorbed and emitted. During fluorescence, the absorption of light excites electrons to a higher electronic state where they remain for about 1-10×10⁻⁸ seconds and then they return to the ground state by emitting a photon of energy. When a population of fluorophores is excited by light of an appropriate wavelength, fluorescent light is emitted. The light intensity can be measured by flurometer or a pixel-by-pixel digital image of the sample.

Fluorescence intensity depends on the efficiency with which fluorescent moieties absorb and emit photons, and their ability to undergo repeated excitation/emission cycles. The intensity of the emitted fluorescent light is a linear function of the amount of fluorophores present. The signal becomes nonlinear at very high fluorophore concentrations.

A wide variety of fluorophores can be attached to the antibodies or probes of the present invention, including but not limited to: green fluorescent protein, orange fluorescent protein, CAL Fluor® Gold 540, CAL Fluor® Orange 560, Quasar® 670, Quasar® 705, 5-FAM (also called 5-carboxyfluorescein; also called Spiro(isobenzofuran-1(3H), 9′-(9H)xanthene)-5-carboxylic acid,3′,6′-dihydroxy-3-oxo-6-carboxyfluorescein); 5-Hexachloro-Fluorescein ([4,7,2′,4′,5′,7′-hexachloro-(3′,6′-dipivaloyl-fluoresceinyl)-6-carboxylic acid]); 6-Hexachloro-Fluorescein ([4,7,2′,4′,5′,7′-hexachloro-(3′,6′-dipivaloylfluoresceinyl)-5-carboxylic acid]); 5-Tetrachloro-Fluorescein ([4,7,2′,7′-tetra-chloro-(3′,6′-dipivaloylfluoresceinyl)-5-carboxylic acid]); 6-Tetrachloro-Fluorescein ([4,7,2′,7′-tetrachloro-(3′,6′-dipivaloylfluoresceinyl)-6-carboxylic acid]); 5-TAMRA (5-carboxytetramethylrhodamine; Xanthylium, 9-(2,4-dicarboxyphenyl)-3,6-bis(dimethyl-amino); 6-TAMRA (6-carboxytetramethylrhodamine; Xanthylium, 9-(2,5-dicarboxyphenyl)-3, 6-bis(dimethylamino); EDANS (5-((2-aminoethyl) amino)naphthalene-1-sulfonic acid); 1,5-IAEDANS (5-((((2-iodoacetyl)amino)ethyl) amino)naphthalene-1-sulfonic acid); DABCYL (4-((4-(dimethylamino)phenyl) azo)benzoic acid) Cy5 (Indodicarbocyanine-5) Cy3 (Indo-dicarbocyanine-3); and BODIPY FL (2,6-dibromo-4,4-difluoro-5,7-dimethyl-4-bora-3a,4a-diaza-s-indacene-3-proprionic acid), ROX, as well as suitable derivatives thereof. Additional examples include, but are not limited to fluorescein, fluorescein chlorotriazinyl, rhodamine green, rhodamine red, tetramethylrhodamine, FITC, Oregon green, Alexa Fluor (e.g. AF488), FAM, JOE, HEX, Texas Red, TET, TRITC, cyanine-based dye and thiadicarbocyanine dye. According to a particular embodiment, the fluorophore is AF488 or EDANS.

In one embodiment, the fluorescent moiety is a quantum dot. Quantum dots are coated nanocrystals fabricated from semiconductor materials in which the emission spectrum is controlled by the nanocrystal size. Quantum dots have a wide absorption spectrum, allowing simultaneous emission of fluorescence of various colors with a single excitation source. Quantom dots can be modified with large number of small molecules and linker groups such as conjugation of amino (PEG) or carboxyl quantum dots to streptavidin (Quantum Dot Corporation, Hayward, Calif., USA).

If the cell specific molecule is fluorescently labeled or the agent that is capable of binding to the cell specific molecule is attached to a fluorescent moiety (i.e. fluorescently labeled antibody or fluorescently labeled probe), particular clusters may be enriched for on the basis of expression of the particular markers by using a fluorescence-activated cell sorter (FACS).

A Flow Cytometer typically consists of a laser light source, flow measurement chamber, and an optical system consisting of lenses, filters, and light detectors. Two photo-multiplier tubes (light detectors), one at 180 degrees and one at 90 degrees to the laser, are used to measure forward (FSC) and right-angle scatter (SSC), respectively. Three fluorescence detectors, each consisting of a filter and photomultiplier tube, are used to detect fluorescence. The three detectors sense green (FL1-530 nm), orange (FL2-585 nm), and red fluorescence (FL3-650 nm). Cells are identified by sort logic applied to all five of the detector signals (FSC, SSC, FL1, FL2, FL3) using a computer.

Exemplary Flow Cytometers that may be used in this aspect of the present invention are manufactured by companies such as Becton Dickinson (USA), Backman Coulter (USA), Partec (Germany).

The FACS machine may be set such that cells of a particular forward scatter and/or side scatter are selected. Forward-scattered light (FSC) is proportional to cell-surface area or size. FSC is a measurement of mostly diffracted light and is detected just off the axis of the incident laser beam in the forward direction by a photodiode. FSC provides a suitable method of detecting particles greater than a given size independent of their fluorescence.

Once the clusters are isolated their transcriptomes are analyzed.

The term “transcriptome” relates to the identity (and in some embodiments also the quantity) of all mRNA molecules produced in one cell or a defined population of cells (e.g. cell cluster). In a particular embodiment, the transcriptome relates to all the mRNA produced in that cell or cluster at a given time point. In another embodiment, the transcriptome relates to the identity of all RNA molecules (including mRNA, rRNA, tRNA, and other non-coding RNA) produced in a cell or a defined population of cells (e.g. cell cluster).

Single Cell Transcriptome Analysis

This method relies on sequencing the transcriptome of a single cell or a single cluster. In one embodiment a high-throughput method is used, where the RNAs from different cells or clusters are tagged individually, allowing a single library to be created while retaining the cell identity of each read. The method can be carried out a number of ways—see for example PCT Publication No. WO2014/108850, Patent Application No. 20100203597 and US Patent Application No. 20180100201, the contents of which are incorporated herein by reference.

In one embodiment, mRNA from single cells (or single clusters) which have been pre-sorted into cell capture plates is barcoded, converted into cDNA and pooled. Subsequently, the pooled sample is linearly amplified by T7 in vitro transcription, and the resulting RNA is fragmented and converted into a sequencing-ready library by tagging the samples with pool barcodes and Illumina sequences during ligation, reverse transcription, and PCR. It will be appreciated that in order to analyze the cells on a single cell basis or single cluster basis, the cells are first distributed into wells, such that only 1 cell or 1 cluster is present per well. The well typically comprises the lysis solution and barcoded poly(T) reverse-transcription (RT) primers for scRNA-seq.

One particular method for carrying out single cell transcriptome analysis is summarized below.

Cells are typically aliquoted into wells such that only one cell is present per well. Cells are treated with an agent that disrupts the cell and nuclear membrane making the RNA of the cell accessible to sequencing reactions.

According to one embodiment, the RNA is amplified using the following in vitro transcription amplification protocol:

(Step 1) contacting the RNA of a single cell with an oligonucleotide comprising a polydT sequence at its terminal 3′ end, a T7 RNA polymerase promoter sequence at its terminal 5′ end and a barcode sequence positioned between the polydT sequence and the RNA polymerase promoter sequence under conditions that allow synthesis of a single stranded DNA molecule from the RNA, wherein the barcode sequence comprises a cell barcode and a molecular identifier;

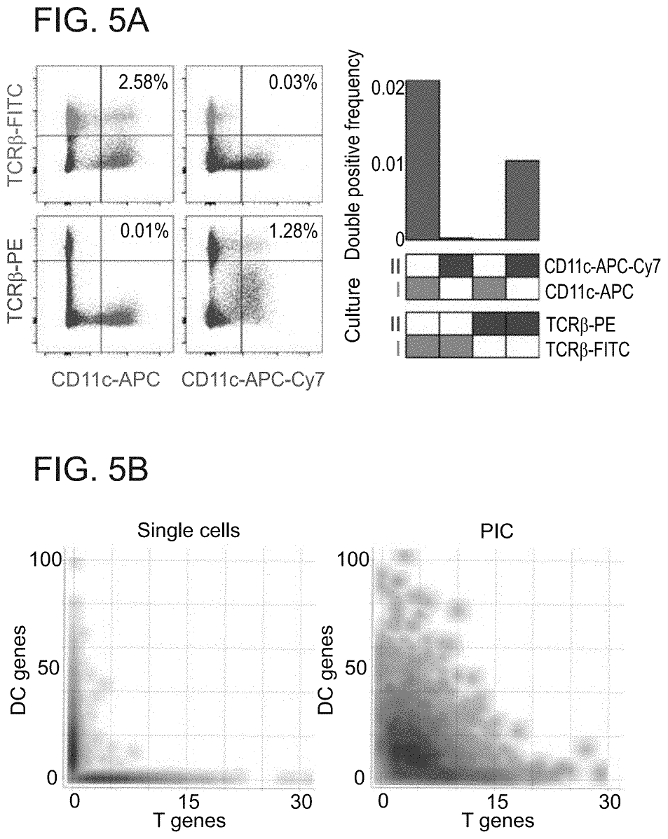

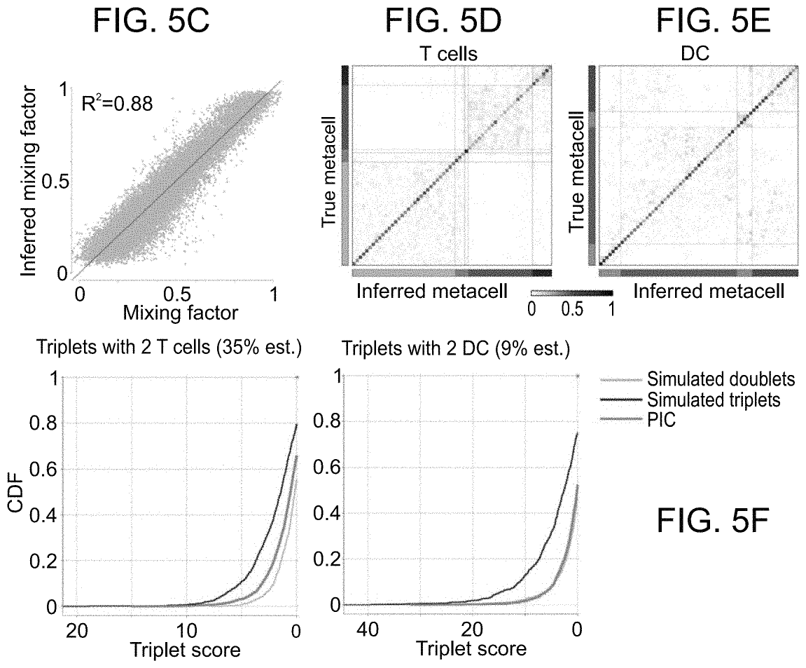

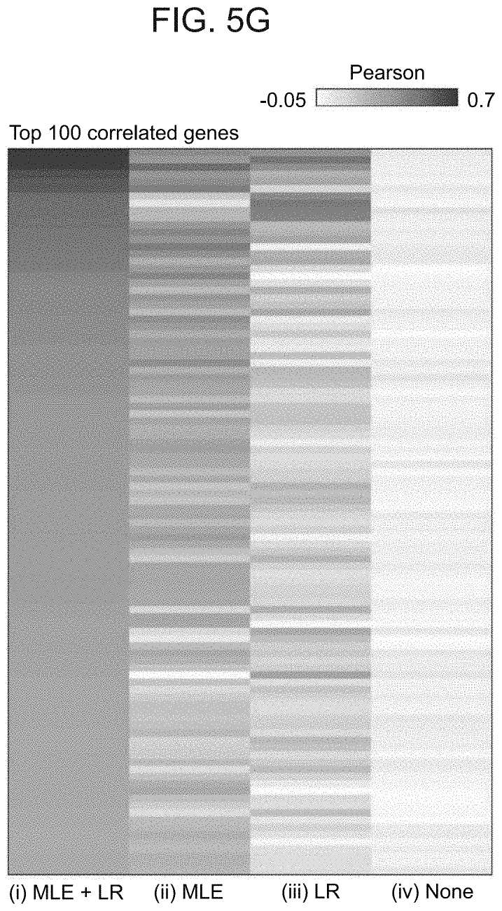

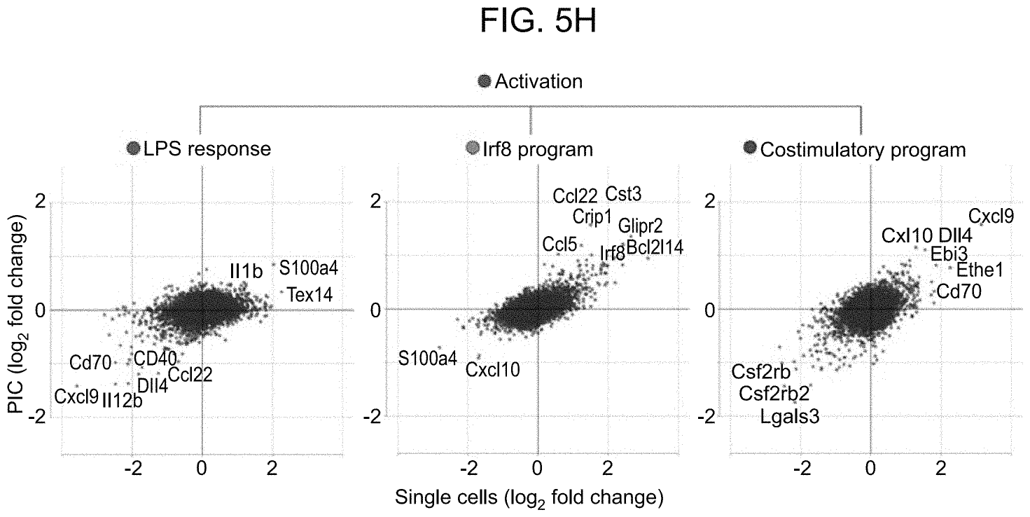

The polydT oligonucleotide of this embodiment may optionally comprise an adapter sequence required for sequencing—see for example FIGS. 5A-H.

RNA polymerase promoter sequences are known in the art and include for example the T7 RNA polymerase promoter sequence.

Preferably the polydT sequence comprises at least 5 nucleotides. According to another embodiment the polydT sequence is between about 5 to 50 nucleotides, more preferably between about 5-25 nucleotides, and even more preferably between about 12 to 14 nucleotides.

The barcode sequence is useful during multiplex reactions when a number of samples are pooled in a single reaction. The barcode sequence may be used to identify a particular molecule, sample or library. The barcode sequence is attached 5′ end of polydT sequence and 3′ of the T7 RNA polymerase sequence. The barcode sequence may be between 3-400 nucleotides, more preferably between 3-200 and even more preferably between 3-100 nucleotides. Thus, the barcode sequence may be 6 nucleotides, 7 nucleotides, 8, nucleotides, nine nucleotides or ten nucleotides.

In one embodiment, the barcode sequence is used to identify a cell type, or a cell source (e.g. a patient).

Molecular identifiers are useful to correct for amplification bias, which reduces quantitative accuracy of the method. The molecular identifier comprises between 4-20 bases. The molecular identifier is of a length such that each RNA molecule of the sample is catalogued (labeled) with a molecular identifier having a unique sequence.

Following annealing of a primer (e.g. polydT primer) to the RNA sample, an RNA-DNA hybrid may be synthesized by reverse transcription using an RNA-dependent DNA polymerase. Suitable RNA-dependent DNA polymerases for use in the methods and compositions of the invention include reverse transcriptases (RTs). RTs are well known in the art. Examples of RTs include, but are not limited to, Moloney murine leukemia virus (M-MLV) reverse transcriptase, human immunodeficiency virus (HIV) reverse transcriptase, rous sarcoma virus (RSV) reverse transcriptase, avian myeloblastosis virus (AMV) reverse transcriptase, rous associated virus (RAV) reverse transcriptase, and myeloblastosis associated virus (MAV) reverse transcriptase or other avian sarcoma-leukosis virus (ASLV) reverse transcriptases, and modified RTs derived therefrom. See e.g. U.S. Pat. No. 7,056,716. Many reverse transcriptases, such as those from avian myeloblastosis virus (AMV-RT), and Moloney murine leukemia virus (MMLV-RT) comprise more than one activity (for example, polymerase activity and ribonuclease activity) and can function in the formation of the double stranded cDNA molecules. However, in some instances, it is preferable to employ a RT which lacks or has substantially reduced RNase H activity.

RTs devoid of RNase H activity are known in the art, including those comprising a mutation of the wild type reverse transcriptase where the mutation eliminates the RNase H activity. Examples of RTs having reduced RNase H activity are described in US20100203597. In these cases, the addition of an RNase H from other sources, such as that isolated from E. coli, can be employed for the formation of the single stranded cDNA. Combinations of RTs are also contemplated, including combinations of different non-mutant RTs, combinations of different mutant RTs, and combinations of one or more non-mutant RT with one or more mutant RT.

Examples of suitable enzymes include, but are not limited to AffinityScript from Agilent or Superscript III from Invitrogen. Preferably the reverse transcriptase is devoid of terminal Deoxynucleotidyl Transferase (TdT) activity.

Additional components required in a reverse transcription reaction include dNTPS (dATP, dCTP, dGTP and dTTP) and optionally a reducing agent such as Dithiothreitol (DTT) and MnCl₂.

The polydT oligonucleotide may be attached to a solid support (e.g. beads) so that the cDNA which is synthesized may be purified.

Annealing temperature and timing are determined both by the efficiency with which the primer is expected to anneal to a template and the degree of mismatch that is to be tolerated.

The annealing temperature is usually chosen to provide optimal efficiency and specificity, and generally ranges from about 50° C. to about 80° C., usually from about 55° C. to about 70° C., and more usually from about 60° C. to about 68° C. Annealing conditions are generally maintained for a period of time ranging from about 15 seconds to about 30 minutes, usually from about 30 seconds to about 5 minutes.

(Step 2): Once cDNA is generated, the cDNA may be pooled from cDNA generated from other single cells (using the same method as described herein above).

The sample may optionally be treated with an enzyme to remove excess primers, such as exonuclease I. Other options of purifying the single stranded DNA are also contemplated including for example the use of paramagnetic microparticles. This may be carried out following or prior to sample pooling.

(Step 3): Second Strand Synthesis.

Second strand synthesis of cDNA may be effected by incubating the sample in the presence of nucleotide triphosphates and a DNA polymerase. Commercial kits are available for this step which include additional enzymes such as RNAse H (to remove the RNA strand) and buffers. This reaction may optionally be performed in the presence of a DNA ligase. Following second strand synthesis, the product may be purified using methods known in the art including for example the use of paramagnetic microparticles.

(Step 4): Following synthesis of the second strand of the cDNA, RNA may be synthesized by incubating with a corresponding RNA polymerase. Commercially available kits may be used such as the T7 High Yield RNA polymerase IVT kit (New England Biolabs).

(Step 5): Prior to fragmentation of the amplified RNA, the DNA may be removed using a DNAse enzyme. The RNA may be purified as well prior to fragmentation. Fragmentation of the RNA may be carried out as known in the art. Fragmentation kits are commercially available such as the Ambion fragmentation kit.

(Step 6): The amplified and fragmented RNA is now labeled on its 3′ end. For this a ligase reaction is performed which essentially ligates single stranded DNA (ssDNA) to the RNA. Other methods of labeling the amplified and fragmented RNA are described in US Application No. 20170137806, the contents of which are incorporated herein by reference. The single stranded DNA has a free phosphate at its 5′end and optionally a blocking moiety at its 3′end in order to prevent head to tail ligation. Examples of blocking moieties include C3 spacer or a biotin moiety. Typically, the ssDNA is between 10-50 nucleotides in length and more preferably between 15 and 25 nucleotides.

(Step 7): Reverse transcription is then performed using a primer that is complementary to the primer used in the preceding step. The library may then be completed and amplified through a nested PCR reaction as illustrated in FIGS. 5A-H.

(Step 8): Amplification

Once the adapter polynucleotide of the present invention is ligated to the single stranded DNA (i.e. further to extension of the single stranded DNA), amplification reactions may be performed.

(Step 9): Sequencing

Methods for sequence determination are generally known to the person skilled in the art. Preferred sequencing methods are next generation sequencing methods or parallel high throughput sequencing methods e.g. Massively Parallel Signature Sequencing (MPSS). An example of an envisaged sequence method is pyrosequencing, in particular 454 pyrosequencing, e.g. based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. Yet another envisaged example is Illumina or Solexa sequencing, e.g. by using the Illumina Genome Analyzer technology, which is based on reversible dye-terminators. DNA molecules are typically attached to primers on a slide and amplified so that local clonal colonies are formed. Subsequently one type of nucleotide at a time may be added, and non-incorporated nucleotides are washed away. Subsequently, images of the fluorescently labeled nucleotides may be taken and the dye is chemically removed from the DNA, allowing a next cycle. Yet another example is the use of Applied Biosystems' SOLiD technology, which employs sequencing by ligation. This method is based on the use of a pool of all possible oligonucleotides of a fixed length, which are labeled according to the sequenced position. Such oligonucleotides are annealed and ligated. Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed and the cycle is repeated. Further examples of sequencing techniques encompassed within the methods of the present invention are sequencing by hybridization, sequencing by use of nanopores, microscopy-based sequencing techniques, microfluidic Sanger sequencing, or microchip-based sequencing methods. The present invention also envisages further developments of these techniques, e.g. further improvements of the accuracy of the sequence determination, or the time needed for the determination of the genomic sequence of an organism etc.

According to one embodiment, the sequencing method comprises deep sequencing.

As used herein, the term “deep sequencing” refers to a sequencing method wherein the target sequence is read multiple times in the single test. A single deep sequencing run is composed of a multitude of sequencing reactions run on the same target sequence and each, generating independent sequence readout.

It will be appreciated that methods which rely on microfluidics can also be used to carry out single cell transcriptome analysis.

Thus, a combination of molecular barcoding and emulsion-based microfluidics to isolate, lyse, barcode, and prepare nucleic acids from individual cells in high-throughput may be used. Microfluidic devices (for example, fabricated in polydimethylsiloxane), sub-nanoliter reverse emulsion droplets. These droplets are used to co-encapsulate nucleic acids with a barcoded capture bead. Each bead, for example, is uniquely barcoded so that each drop and its contents are distinguishable. The nucleic acids may come from any source known in the art, such as for example, those which come from a single cell, a pair of cells, a cellular lysate, or a solution. The cell is lysed as it is encapsulated in the droplet. To load single cells and barcoded beads into these droplets with Poisson statistics, 100,000 to 10 million such beads are needed to barcode about 10,000-100,000 cells. In this regard there can be a single-cell sequencing library which may comprise: merging one uniquely barcoded mRNA capture microbead with a single-cell in an emulsion droplet having a diameter of 75-125 μm; lysing the cell to make its RNA accessible for capturing by hybridization onto RNA capture microbead; performing a reverse transcription either inside or outside the emulsion droplet to convert the cell's mRNA to a first strand cDNA that is covalently linked to the mRNA capture microbead; pooling the cDNA-attached microbeads from all cells: and preparing and sequencing a single composite RNA-Seq library, as described herein above. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; Zheng, et al., 2016, “Haplotyping germline and cancer genomes with high-throughput linked-read sequencing” Nature Biotechnology 34, 303-311; and International patent publication number WO 2014210353 A2, all the contents and disclosure of each of which are herein incorporated by reference in their entirety.

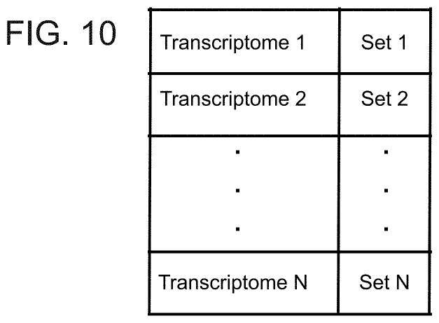

As mentioned, the method comprises accessing a computer readable medium storing a library having a plurality of entries, each entry having a predicted transcriptome of a cell cluster and a set of identities of known cell types forming the cell cluster. A representative example of a library which can be stored on computer readable medium according to some embodiments of the present invention is illustrated in FIG. 10. In the illustrated example, the library contains N entries, enumerated 1 through N.

According to another aspect of the present invention, there is provided a method of identifying cell members of a cell cluster in a tissue of interest:
(a) isolating a plurality of single cells of different cell types from the tissue of interest on the basis of expression of a unique cell marker;
(b) determining the transcriptomes of said single cells of different cell types;
(c) obtaining predictions of the transcriptomes of a plurality of clusters of non-identical cells of said plurality of single cells from said transcriptomes of said single cells, each of said plurality of clusters comprising a unique combination of cells, wherein the predictions are based on the null hypothesis that the transcriptome of a cluster is the sum of a transcriptome of the individual cells of the cluster when in a single cell state;
(d) isolating a cluster of cells from said tissue;
(e) performing transcriptome analysis of said cluster of cells so as to obtain the true transcriptome of said cluster;
(f) comparing said predictions to said true transcriptome; and
(g) identifying the members of the cell cluster by selecting the transcriptome prediction that has the closest identity to said true transcriptome.

Particular step of the multi-step method will be described in further detail herein below. Steps that relate to the isolation of cells and clusters and to the analysis of the cell clusters have been described herein above.

(a) isolating a plurality of single cells of different cell types from the tissue of interest on the basis of expression of a unique cell marker.

Isolation of cells has been described herein above. In this step, care should be taken that cells are isolated to the single cell level and care should be taken such that the final suspension of cells should be devoid of cell clusters. Preferably, less than 20% of the cells present in the final suspension are present as cell clusters, less than 10% of the cell present in the final suspension are present as cell clusters, less than 8% of the cell present in the final suspension are present as cell clusters, less than 5% of the cell present in the final suspension are present as cell clusters, less than 4% of the cell present in the final suspension are present as cell clusters, less than 3% of the cell present in the final suspension are present as cell clusters, less than 2% of the cell present in the final suspension are present as cell clusters, and even less than 1% of the cell present in the final suspension are present as cell clusters.

Unique cell markers are described herein above. The marker may be unique for the cell type or may be a pan-cell marker. Alternatively, the marker may be unique for a cell state.

(c) obtaining predictions of the transcriptomes of a plurality of clusters of non-identical cells of said plurality of single cells from said transcriptomes of said single cells, each of said plurality of clusters comprising a unique combination of cells, wherein the predictions are based on the null hypothesis that the transcriptome of a cluster is the sum of a transcriptome of the individual cells of the cluster when in a single cell state. In this step, grouping of cells isolated from the single cell states of both contributing cell states into homogeneous transcriptional entities may be performed using conventional analytical tools for cell clustering (based, for example, on k-nearest neighbors algorithms, e.g. MetaCell, Seurat, etc.). This clustering of single cells may serve as a background model to derive for each cell cluster two identities which represent the most likely pair of cells that gave rise to this specific cell cluster. Cell clusters may be modeled as a linear mixture of pairs of contributing single cells. Each contributing single cell (e.g. T cells or DC) belongs to a group of cells from the respective background models calculated over the single cell populations, and its gene expression may be sampled from the multinomial probability distribution derived from that group. The mixing factor assigned for each cell cluster, denotes the fraction of transcripts contributed by one of the contributing single cells. This may be performed in a two-step approach:

First, a linear regression model may be trained on synthetic cell clusters to infer the mixing for each real cell cluster. Second, all possible combinations of single cell groups from single cell populations may be deduced and the expected gene expression distributions of these mixtures may be calculated. A maximum likelihood estimator may be applied on each cell cluster, to derive two groups of cells whose mixture is most likely to give rise to it.

(f) comparing said predictions to said true transcriptome;

The expected expression of each gene in a certain cell cluster may be calculated as the weighted sum of the transcriptional contribution from one single cells (which can be estimated from the characteristic multinomial distribution of the assigned group of cells), and the contribution from the other contributing part. This sum may be weighted by the mixing factor inferred for each PIC. Statistical test for hypothesis testing χ2 test, or Mann-Whitney test) can then be used to systematically scan for genes whose true transcription values diverge from expected in specific groups of cell clusters.

It will be appreciated that once the members of the cell cluster are identified, the present inventors propose identifying which genes are regulated by cell clustering. This can be effected by comparing the amount of mRNA of particular genes present in the single cells with the amount of mRNA of those particular genes present in the same cell when it is part of a cluster. If the amount of mRNA is changed beyond a particular level (e.g. increased by more than 10%, 20% 30%, 40%, 50%, 60%, 70%, 80%, 90% 100% or more in a statistically supported manner) in the cell when it is part of a cluster, over then amount of that gene when it is present as a single cell, then it can be deduced that clustering positively regulates that gene. On the other hand, if the amount of mRNA is changed beyond a particular level (e.g. decreased by more than 10%, 20% 30%, 40%, 50%, 60%, 70%, 80%, 90% 100% or more) in the cell when it is part of a cluster, over then amount of that gene when it is present as a single cell, then it can be deduced that clustering negatively regulates that gene.

In a particular embodiment, identifying which genes are regulated by cell clustering is carried out as follows:
(a) obtaining data of the transcriptome of a first cell of the cluster, wherein said transcriptome is obtained when said first cell is in a single-cell state;
(b) obtaining data of the transcriptome of a second cell of the cluster, wherein said transcriptome is obtained when said second cell is in a single-cell state, wherein said first cell and said second cell express non-identical cell surface markers;
(c) obtaining a prediction of the transcriptome of a cluster of said first cell and said second cell from said data of the transcriptome of said first cell in a single-cell state and said data of the transcriptome of said second cell in a single cell state, using the null hypothesis that said transcriptome of said cluster is the combination of said transcriptome of said first cell in said single cell state and said transcriptome of said second cell in said single cell state;
(d) isolating a cluster of cells which comprise said first cell and said second cell from a heterogeneous population of cells;
(e) performing transcriptome analysis of said cluster of cells so as to obtain the true transcriptome of said cluster; and
(f) comparing said prediction to said true transcriptome, wherein expression of a gene of said true transcriptome that is statistically significantly altered is indicative of a gene that is regulated by cell clustering.

Additionally, or alternatively, one the member of particular cell clusters are identified, the method can be used to determine the abundance or presence of a cell cluster of a particular combination present in a tissue of interest. This information may serve as an indication of a disease state i.e. may be used to diagnose a disease. Exemplary diseases which may be diagnosed include cancer, infectious diseases, fibrosis and immune diseases (including autoimmune diseases).

As used herein, the term “diagnosing” refers to determining presence or absence of the disease in the subject, classifying the disease, determining a severity of the disease, monitoring disease progression, forecasting an outcome of a pathology and/or prospects of recovery and/or screening of a subject for the disease.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

PIC-Seq Application on T Cells and Dendritic Cells (DC)

Materials and Methods

Mice: C57BL/6 WT female mice and day 0 neonate mice were obtained from Harlan. TCR-transgenic OT-II mice [harboring ovalbumin (OVA)—specific CD4⁺ T cells] and RFP-FOXP3 mice also used.

In vitro cultures: Cells were isolated from spleens of C57BL/6 8-week-old mice. Splenocytes were washed and suspended in red blood lysis buffer (Sigma-Aldrich) and DNase I (0.33 U/ml, Sigma-Adrich), incubated for 5 min at room temperature, washed twice with cold PBS, filtered through a 70 μm cell strainer, centrifuged at 400 g, 5 min, 4° C. and then resuspended in ice cold sorting buffer (PBS supplemented with 0.2 mM EDTA pH8 and 0.5% BSA). DC derived from spleen tissues were enriched from the single cell suspension first by negative magnetic selection for CD19, and second by positive magnetic selection for CD11c. Briefly, cells were incubated with CD19 MicroBeads (CD19 MicroBeads; Miltenyi Biotech) for 15 minutes at 4° C., washed and run through a MACS column (Miltenyi Biotech). Negative fraction of cells was collected for further incubation with CD11c MicroBeads (CD11c MicroBeads Ultrapure; Miltenyi Biotech) for 10 minutes at 4° C., and positive fraction of CD11c⁺ cells was collected. Following cell counting 2×10⁶ DC were seeded in 6-well plate in 3 ml of standard media RPMI-1640 supplemented with 10% FCS, 1 mM 1-glutamine, 100 U/ml penicillin, 100 mg/ml streptomycin (Biological Industries), for 2 h activation with 100 ng/ml lipopolysaccharide (LPS; Sigma-Aldrich) and 2 ug/ml OVA 323-339 (InvivoGen), at 37° C. In parallel, we isolated CD4⁺ T cells from spleens of OT-II mice. Again, cells were washed and suspended in red blood lysis buffer (Sigma-Aldrich) and DNase I (0.33 U/ml, Sigma-Adrich), incubated for 5 min at room temperature, washed twice with cold PBS, filtered through a 70 μm cell strainer and centrifuged at 400 g, 5 min, 4° C. CD4⁺ T cells were enriched by the CD4+ T cell Isolation Kit, according to manufacture instructions (Miltenyi Biotech). Briefly, splenocytes were incubated with biotin-antibody cocktail for 5 min, and afterwards anti-biotin microbeads were added for additional 10 min incubation. We collected the CD4⁺ T cells, which were the unlabeled fraction. For culture experiments CD11c⁺ DC and CD4⁺ T cells were plated on 96-well U-shape bottom, tissue culture plates. Co-cultured and mono-cultured cells were seeded in concentration of 1×10⁶ cells/ml (1:1 ration in co-cultures), and harvested at different time points following cell plating (3 h, 20 h, 44 h). All cultures were done in the standard media RPMI-1640 supplemented with 10% FCS, 1 mM 1-glutamine, 100 U/ml penicillin, 100 mg/ml streptomycin (Biological Industries).

Transwell in vitro assay: To assess whether cell activation was a result of physical interaction between CD11c⁺ DC and CD4⁺ T cells or of secreted factors in the culture, we plated the cells on 12-well tissue culture plates with inserts of 0.4 μm pore size. For co-culture samples, each cell type was seeded in plate bottom and in the insert. Co-cultured and mono-cultured cells were seeded in concentration of 1×10⁶ cells/ml (1:1 ration in co-cultures), and harvested 20 h following cell plating. All cultures were done in the standard media RPMI-1640 supplemented with 10% FCS, 1 mM 1-glutamine, 100 U/ml penicillin, 100 mg/ml streptomycin (Biological Industries).

Preparation of Antigen: N. brasiliensis (Nb) infective L3 larvae were prepared, washed in sterile PBS and killed by three freeze-thaw cycles as described⁶⁰. For fluorescent labelling of antigens, non-viable Nb were incubated for 10-15 minutes in 0.05M NaHCO3 buffer and 0.1 mg AF488 NHS Ester (Molecular Probes, Invitrogen) and then washed with 0.1M Tris buffer.

Immunization and In vivo Treatments: Mice were anesthetized and antigen was administered by intradermal injection into the ear pinna. PBS was injected into the ear pinna of control animals. Experiments were performed 48 h following antigen injection.

Tissue dissociation and single cell sorting: To achieve single cell suspensions, auricular LN were digested in IMDM (Sigma-Aldrich) media. For mild dissociation tissues were supplemented with Liberase-TL (100 μg/ml, Roche) and DNase-I (100 μg/ml, Roche), and incubated with frequent agitation at 37° C. for 20 min. For strong dissociation tissues were supplemented with Liberase-TL (200 μg/ml, Roche) and DNase-I (100 μg/ml, Roche), and incubated with frequent agitation at 37° C. for 40 min.

In order to achieve epithelial and immune cell PIC from the neonate lung, neonates were euthanized by laying on a frozen surface, and then perfused by injection of cold PBS via the right ventricle prior to lung dissection. Lung tissues were dissected from mice and were homogenized using lung dissociation kit (Miltenyi Biotec), including 15 min incubation at 37. In additional tested dissociation protocol cells were supplemented with DMEM/F12 medium (Sigma-Aldrich) containing Elastase (3 U/ml, Worthington) and DNase (0.33 U/ml, Sigma-Aldrich) incubated with frequent agitation at 37° C. for 15 min.

After all dissociation procedures, cells were washed with dissociation media, filtered through a 100 μm cell strainer, and centrifuged at 380 g, 5 min, 4° C.

Flow cytometry and sorting: Cells were suspended in ice cold sorting buffer (PBS supplemented with 0.2 mM EDTA pH8 and 0.5% BSA) supplemented with anti-mouse CD16/32 (BD Bioscience) to block Fc receptors prior to labelling with fluorescent antibodies against cell surface epitopes. Samples were stained using the following antibodies: eFluor450-conjugated TER-119 and Pacific blue-conjugated CD19 were purchased from eBioscience, PerCP Cy5.5-conjugated TCRβ, PE-conjugated TCRβ, FITC-conjugated TCRβ, APC-Cy7-conjugated TCRβ, APC-conjugated CD11c, APC-Cy7-conjugated CD11c, PE-conjugated CD160, Biotin-conjugated ICOS, FITC-conjugated CD326, and PerCP Cy5.5-conjugated streptavidin were purchased from Biolegend. Prior to sorting, cells were stained with DAPI for evaluation of live/dead cells. For the sorting of T-DC PIC samples were gated for TCRβ⁺CD11c⁺, for sorting of T cells samples were gated for TCRβ⁺, and for the isolation of DC, samples were gated for CD11c⁺, after exclusion of dead cells (DAPI⁻), erythrocytes (TER119−) and B cells (CD19−). For the sorting of immune-epithelial PIC in the developing lung, cells were stained for EPCAM (CD326) and CD45.

For evaluation of protein levels of DLL4 expressed by Ag⁺PIC, we performed cell surface staining of PE-conjugated DLL4 (Biolegend).

Cell populations were sorted with SORP-aria (BD Biosciences, San Jose, Calif.) or with ARIA-III instrument (BD Biosciences, San Jose, Calif.), and analyzed using BD FACSDIVA software (BD Bioscience) and FlowJo software (FlowJo, LLC).

Isolated live cells were single-cell sorted into 384-well cell capture plates containing 2 μL of lysis solution and barcoded poly(T) reverse-transcription (RT) primers for single-cell RNA-seq^{16, 61}. Four empty wells were kept in each 384-well plate as a no-cell control during data analysis. Immediately after sorting, each plate was spun down to ensure cell immersion into the lysis solution, and stored at −80° C. until processed.

Visualization of PIC by sorting and confocal microscopy: Cells were purified from auricular LN of naïve WT and RFP-FOXP3 mice. Following exclusion of dead cells, erythrocytes and CD19 expressing B cells, CD11c⁺TCRβ⁺ PIC were gated using the antibodies PE-conjugated TCRβ and APC-conjugated CD11c (Biolegend), and bulk sorted to a tube containing 50 μl sorting buffer. Sorted PIC were directly fixed for 10 min with paraformaldehyde (4% PFA; Santa Cruz) and then directly permeabilized by addition of triton X-100 (0.1%; Sigma-Aldrich) for 5 min on ice. For detection of cell nuclei, DAPI was added for 30 sec. PIC were washed and applied to slides, mounted with Immu-mount (Thermo Scientific), and sealed with cover-slips. Microscopic analysis was performed using a laser-scanning confocal microscope (Zeiss, LSM880). Images were acquired and processed by Imaris software (Bitplane, Zurich, Switzerland).

Immunohistochemistry: For spatial examination, frozen sections of auricular LN were taken at 48 h following Nb infection with double dose or PBS injection to the ear pinna of the mice. LN were fixed in 4% PFA solution for 4 h, and then transferred to 30% sucrose solution for 2 days. Tissues were embedded in O.C.T.™ (Sigma-Aldrich) and 10 μm sections were performed by LEICA CM1950 machine. For visualization of T cells and DC, sections were first blocked with a blocking buffer solution (5% FBS, 1% BSA, 0.2% triton) for 2 h at room temperature. Sections were washed with PBST (0.01% tween-20; Sigma-Aldrich) three times and then blocked with 0.1% Sudan Black B (Sigma-Aldrich) solution for 20 min at room temperature. Sections were washed three time with PBST and incubated with antibodies over-night at 4° C. Antibodies that were used are: PE-conjugated TCRβ and APC-conjugated CD11c (1:50; Biolegend). Sections were washed three times with PBST, DAPI dye was added for 10 min to detect cell nuclei, and then washed with PBST. Sections were mounted with SlowFade™ (Invitrogen) and sealed with cover-slips. Microscopic analysis was performed using a laser-scanning confocal microscope (Zeiss, LSM880). Images were acquired and processed by Imaris software (Bitplane, Zurich, Switzerland).

MARS-seq Library preparation: Single-cell libraries were prepared as previously described^{16, 29}. In brief, mRNA from cell sorted into cell capture plates were barcoded and converted into cDNA and pooled using an automated pipeline. The pooled sample is then linearly amplified by T7 in vitro transcription, and the resulting RNA is fragmented and converted into a sequencing-ready library by tagging the samples with pool barcodes and illumina sequences during ligation, RT, and PCR. Each pool of cells was tested for library quality and concentration is assessed as described earlier.

MARS-seq low level data processing: scRNA-seq libraries (pooled at equimolar concentration) were sequenced on an Illumina NextSeq 500 at a median sequencing depth of 23,998 reads per cell. Sequences were mapped to the mouse genome (mm10), demultiplexed, and filtered as previously described (Jaitin et al., 2014) with the following adaptations. Mapping of reads was done using HISAT (version 0.1.6); reads with multiple mapping positions were excluded. Reads were associated with genes if they were mapped to an exon, using the UCSC genome browser for reference. We estimated the level of spurious UMIs in the data using statistics on empty MARS-seq wells. Cells with less than 500 UMI, more than 500,000 UMI, or with more than 20% mitochondrial genes were excluded from analysis.

PIC-seq algorithm: PIC-seq allows the deconvolution of heterotypic particles of physically interacting cells, by assigning each PIC with the transcriptional identity of its contributing partners, based on the background singlets model. The computational framework behind PIC-seq relies on several assumptions, derived from the experimental approach:
(1) PIC are composed of cells derived from two distinct single-cell populations (A and B).
(2) The contributing partners of each PIC can be approximated as a sampling of two representative cells from the background singlets datasets of populations A and B.
(3) Most of the PIC transcripts can be modeled as a linear combination of the contributing partners' transcripts, mixed at a specific ratio (the mixing factor α).

A computational framework (the PIC-seq algorithm) is presented based on these underlying assumptions. The algorithm receives as input a genes-over-cells expression matrix of PIC (P_{gXc}), and of two background single-cell, non-conjugated populations (A_{gXc} and B_{gXc}). In addition, it receives two metacell background models, mc_{A} and mc_{B}, which assign a meta cell identity to each single cell from populations A and B, respectively. The algorithm returns as output three values for each PIC—the PIC metacell assignment of its contributing partners from populations A and B (mc_{A}^{P}, mc_{B}^{P}); and the mixing factor a. Thus, each PIC can be described as a linear mixture of two cells whose gene expression vectors are sampled from their corresponding metacells:

uₚ=α·u_{A}+(1−α)·u_{B};u_{A}˜P(mc_{P}^{A}),u_{B}˜P(mc_{B}^{P})

The PIC-seq algorithm operates in two steps. First, we apply a linear regression model trained on synthetic PIC to infer a for each PIC. Second, we construct all possible combinations of metacells from populations A and B mixed by a and calculate the expected gene expression distributions of these mixtures. A maximum likelihood estimator is now applied on each PIC, choosing mc_{A}^{P}, mc_{B}^{P} whose mixture is most likely to give rise to the PIC. In the next paragraphs we describe in detail how α, mc_{A}^{P} and mc_{B}^{P} are inferred. Importantly, PIC-seq algorithm first models each PIC as a heterotypic doublet (one cell from population A and one from B). Our approach to account for triplets in PIC-seq data is described below.

Simulating synthetic PIC: In order to train a linear regression model for the mixing factor, we first simulate N artificial PIC by pairing and pooling single cells taken from populations A and B. Cells from A and B were sampled from the same condition (e.g. same timepoint or infection status) and preserved condition proportions.

Combined pairs of cells were down-sampled to a predefined total number of UMI (numis), while preserving differences in cell size within each pair.

$${simU}_{a , b} = downsample ( a , numis \cdot \frac{\vert {U}_{a} \vert}{( \vert {U}_{a} \vert + \vert {U}_{b} \vert )} ) + downsample ( b , numis \cdot \frac{\vert {U}_{b} \vert}{( \vert {U}_{a} \vert + \vert {U}_{b} \vert )} )$$

Where |U_{a/b}| denotes the number of UMI in cell a and b, respectively. For each simU_{a,b} we denote the mixing factor α as the fraction of UMI taken from a:

$$α = \frac{\vert {U}_{a} \vert}{( \vert {U}_{a} \vert + \vert {U}_{b} \vert )}$$

Estimating the mixing factor: We developed a linear regression strategy that estimates a from gene expression, and trained it on a set of artificial PIC. Our model assumes that since populations A and B are mutually exclusive, and each population's heterogeneity can be described by a unique set of lineage-specific genes, the rich information of lineage-specific genes in each PIC can be used to estimate α.

First, we selected a set of genes as features for the model. These include the genes used to generate the metacell models, as well as a set of genes whose correlation with UMI count is highest in one of the populations.

We implemented linear regression with the glmnet R package. We executed lasso with 10-fold cross validation on the artificial PIC, with the down-sampled feature genes as independent variables, and a as the dependent variable. We used the resulting model to obtain estimates of a for the down-sampled matrix of PIC.

Assigning PIC Contributing Partners with MLE

We designed an Maximum Likelihood Estimator (MLE) that assigns each PIC with the metacell identities of its contributing partner cells, derived from populations A and B (mc_{A}^{P}, mc_{B}^{P}). Given α, mc_{A}^{P} and mc_{B}^{P} the log likelihood of a gene expression vector uₚ to be derived from an α-mixture of cells from the two metacells can be described by mixing two multinomial distributions:

$$log ( L ) = log ( {M}_{p} ) + {\sum }_{g ∈ G} {u}_{p}^{g} \cdot log ( α \cdot {P}_{m {c}_{A}^{P}}^{g} + ( 1 - α ) \cdot {P}_{m {c}_{B}^{P}}^{g} )$$

Where Mₚ is the multinomial coefficient, G denotes a set of genes, and

$${P}_{m {c}_{A}^{P}}^{g} and {P}_{m {c}_{B}^{P}}^{g}$$

are probability vectors, signifying the probability to draw a UMI of a gene (g) in metacells mc_{A}^{P} and mc_{B}^{P}, respectively.
The PIC-seq algorithm computes for each PIC gene expression vector (uₚ) and its mixing factor (a) the multinomial distributions of α-mixtures of all combinations of metacells from A and B, uses these distributions to calculate the log likelihood values of uₚ, and assigns each PIC with the maximum likelihood contributing partners.
Mₚ is computed with the Stirling's approximation. Multinomial probability vectors for metacells P_{mc} were extracted from the geometric means of all cells in each metacell, as previously described³². Genes with less than 10 total UMI were discarded. A regularization constant (10⁻⁴) was added to all multinomial distributions to avoid null probabilities.

Estimation and Filtration of Triplets

In every PIC-seq dataset, a certain fraction of PIC may be composed of triplets, which, if not accounted for, may influence analysis and lead to biases. We extended PIC-seq design to computationally estimate the fraction of triplets in a dataset and filter PIC identified as triplets. We first run basic PIC-seq to determine α, mc_{A}^{P} and mc_{B}^{P} for each PIC and the maximum log likelihood values when the PIC is modeled as a doublet: ll_{Doublet}. We then calculate the maximum log likelihood of the PIC when modeled as a triplet composed of two cells from population A and one from population B: ll_{Triplet} (we model the opposite scenario—to cells from population B—separately). To reduce the model space, we treat α and mc_{B}^{P} as invariable, and produce all possible α-mixtures of mc_{B}^{P} and two metacells from population A. The mixing proportions of the two metacells from population A are determined empirically for each pair of metacells by the median UMI count of cells in each metacell. Log likelihood for a triplet model is computed similarly to a doublet model. We then define the triplet score of a PIC, S=ll_{Triplet}−ll_{Doublet}, as the log fold change between the likelihoods of it being a triplet and a doublet.

We used synthetic doublets and triplets to derive empirical distributions of the triplet score for doublets (S_{Doublet}) and triplets (S_{Triplet}). given a triplet score s and a prior triplet rate R, the posterior probability of a PIC being a triplet is:

$$P ( Triplet \vert s ) = \frac{{S}_{Triplet} ( s ) \cdot R}{{S}_{Triplet} ( s ) \cdot R + {S}_{Doublet} ( s ) \cdot ( 1 - R )}$$

And the posterior expected triplet frequency is:

$$\frac{1}{N} {\sum }_{i = 1}^{N} P ( Triplet \vert {s}_{i} ) .$$

We use these equations to derive an estimation of the triplet rate, choosing the posterior probability whose deviation from the prior probability is minimal.
Importantly, our power to detect triplets is limited to cases where the two A cells are derived from transcriptionally distinct metacells (heterotypic triplets). We account for this fact by limiting our model space to contain only metacells that share less k-nearest neighbors than expected.

Downstream Analysis Execution

In the following sections we describe how PIC-seq was executed on three distinct datasets. Dataset I includes all single cells and PIC from the in vitro experiments (mono-culture, co-culture and transwell). Dataset II includes all single cells and PIC from the in vivo infection models (Nb infected and PBS control). Dataset III includes single cells and PIC from the postnatal lung. To get a full representation of transcriptional state in the postnatal lung, we augmented dataset III with CD45⁺ and CD45⁻ single cells from days 0-2 PN, as well as day 2 PN CD45⁺FcεR1a⁺cKit⁻ basophils from a previous publication²⁴.

Metacell Covers of Singlets and PIC

For each dataset, we used the MetaCell package (Baran et al., 2018) to build two separate metacell covers, one for all single cells (the background singlet model), and one for all PIC. We first removed mitochondrial genes, ERCC, and predicted genes with high abundance (Gm42418, Gm26917, and Gm29216). We then filtered cells with less than 500 UMI, more than 500,000 UMI or total fraction of mitochondrial gene expression exceeding 20%. For datasets I and II, we further discarded cells with high joint expression of B cell genes (Cd79b, Cd19, Vpreb3, C3; more than 7 UMI total).

Gene features for metacell covers were selected using the parameter Tᵥₘ=0.2, total umi> 20, and more than 3 UMI in at least 3 cells (using the functions mcell_gset_filter_varmean, and mcell_gset_filter_cov; for dataset III parameters were Tᵥₘ=0.3, total umi >50, and 4 UMI). We excluded gene features associated with the cell cycle and MHC-II via a clustering approach (using the functions mcell_mat_rpt_cor_anchors and mcell_gset_split_by_dsmat). To this end we first identified all genes with a correlation coefficient of at least 0.1 for one of the anchor genes Top2a, Mki67, Pcna, Mcm4, Cdk1, Ube2c (cell cycle), and H2-Aa (MHC-II). We then hierarchically clustered the correlation matrix between these genes (filtering genes with low coverage and computing correlation using a down-sampled UMI matrix) and selected the gene clusters that contained the above anchor genes. We thus retained 120 (dataset I), 315 (dataset II) and 522 (dataset III) genes as features. We used metacell to build a kNN graph, perform boot-strapped co-clustering (500 iterations; resampling 70% of the cells in each iteration), and derive a cover of the co-clustering kNN graph (K=30 for dataset I and K=70 for datasets II and III). Outlier cells featuring gene expresssion higher than 4-fold than the geometric mean in the metacells in at least one gene were discarded (75 cells in dataset I, 35 in II and 123 in dataset III, using function mcell_mc_split_filt).

Annotation of the singlets background model of dataset I was performed by thresholding over expression of correlated gene modules. Annotation of the singlets background model of dataset II was performed by expression of marker genes according to literature, as described in Table 1:

TABLE 1

| | | | Fold |
|---|---|---|---|
| Cell type | Marker | Priority | change |
| Naïve T | Trbc2 | 1 | 0.5 |
| Naïve T | Cd3e | 1 | 0.5 |
| CD8T | Cd8b1 | 2 | 3 |
| CD8T | Cd8a | 2 | 3 |
| Treg | Icos | 2 | 3.5 |
| Treg | Tigit | 2 | 2.2 |
| Treg | Il2ra | 2 | 2.5 |
| Activated T | Nme1 | 4 | 2 |
| CD8 mem T | Il2rb | 4 | 4 |
| CD8 mem T | Ctla2a | 4 | 2.6 |
| pDC | Siglech | 5 | 10 |
| Migratory DC | Tmem123 | 4 | 7 |
| cDC1 | Wdfy4 | 1 | 4 |
| cDC2 | Csf1r | 3 | 2 |
| cDC2 | Fcer1g | 3 | 2.2 |
| cDC2 | Cd209a | 5 | 4 |
| Monocyte | Lyz2 | 5 | 10 |
| NK | Gzma | 5 | 4 |
| B | Cd79b | 5 | 3 |

Metacells were assigned to the T or DC/myeloid lineages based of expression of Trbc2 and Fscn1lCst3. In Dataset I, PIC metacells expressing high levels of Klrc1 or Igkc were annotated as Natural Killer or B cells, and were discarded from further analysis. Annotation of dataset III singlets was performed as previously described²⁴.

Correlated gene modules: To derive correlated gene modules in T cells from dataset I, we defined a set of anchor genes by choosing top 5 differential genes in each T associated metacell whose mean expression is at least 2-fold higher than the median across all T metacells. We then extracted all genes with a correlation coefficient of at least 0.13 for at least one anchor gene, resulting in a list of 273 genes (using the function mcell_mat_rpt_cor_anchors). We then hierarchically clustered the pairwise correlation matrix of these genes, and used the cutree function with K=10 to retrieve modules (using the function mcell_gset_split_by_dsmat). We repeated this procedure for DC metacells (with correlation threshold of 0.2), resulting in a list of 238 genes divided into 20 modules.

We computed the pooled size-normalized module expression of each cell, and interrogated the median module expression across meta-cells. Threshold values were manually determined to organize meta-cells into 5 T and 4 DC subtypes.

Running the PIC-seq algorithm: To derive genes that would serve as features for the linear regression, we selected single cells collected from only one experiment (to account for different library sizes between experiments), and removed cells with high cell cycle activity (at least 32 UMI of cell cycle genes). We then computed genes' correlation with each cell's UMI count, for T cells and DC separately. We selected the top 100 most UMI-correlated genes in T cells and in DC, as well the genes used as features in the metacell cover (also removing ribosomal genes). Overall, we selected 289 genes for dataset I, 448 genes for dataset II, and 647 genes for dataset III.

In all datasets, both synthetic and real PIC matrices were downsampled to 1000 UMI per cell (numis=1000). For the linear regression, R² values for estimating the mixing coefficient over the synthetic PIC were 0.88 for dataset I (FIGS. 5A-H; 10-fold cross validation), 0.77 for dataset II, and 0.87 for dataset III.

To derive genes for computing the MLE assignment, we chose the top 20 differential genes in each T (or DC) associated metacell whose mean expression is at least 2-fold higher than the median across all T (or DC) metacells. We combined this set of genes with the genes used as features in the metacell cover, but discarded genes which are highly differential both in the T and DC metacell models (more than 2-fold enrichment in at least on meta-cell in both populations), as well as ribosomal genes. Overall, we selected 458 genes in dataset I, 233 in dataset II, and 443 in dataset III. We then calculated the multinomial distribution of each metacell over these derived sets of genes (such that each probability vector sums to 1).

We validated PIC-seq assignments in several ways: We computed the error in assignments over 5,000 synthetic PIC, and showed that wrong assignments from one T or DC subset to another are low (FIGS. 5A-H and 7A-J). For dataset I, we also provide a leave 10% out cross-validation, where each gene's Pearson correlation between the observed and expected PIC profiles (as described below) is computed when that same gene was part of the left out gene set (FIGS. 5A-H). We show that PIC-seq (i) outperforms three partial models: (ii) where metacell assignments are maintained but α is set to 0.5, (iii) where α is maintained but metacell assignments are shuffled across PICs, and (iv) a null model where both α and metacell assignments are arbitrary.

Estimation of triplet rates was executed as described above for both datasets (FIGS. 5A-H and 7A-J). In dataset II, since estimated triplet rates were low (4% for 2T and on DC and 3% for the opposite scenario), we filtered the top 4% (3%) PIC whose triplet score was highest. Simulations on mixtures of synthetic doublets and triplets mixed at these ratios suggest approximately 20% of all triplets can be filtered this way (FIGS. 7A-J).

To discard triplets in dataset III containing cells other than epithelium and immune cells, we empirically derived gene sets enriched in other cell populations present in the singlet dataset (endothelium, fibroblasts and erythrocytes). We then scored PIC according of their pooled expression of these gene sets and discarded cells with high expression of at least one of these programs.

Comparing Observed and Expected Expression

PIC-seq assigns each PIC with a triplet of α, mc_{A}^{P} and mcB^{P}. Based on these estimates, it is possible to reconstruct the expected levels of a gene in a PIC:

$$exp ( {u}_{p}^{g} ) = mumis \cdot α \cdot {P}_{m {c}_{A}^{P}}^{g} + numis \cdot ( 1 - α ) \cdot {P}_{m {c}_{B}^{P}}^{g}$$

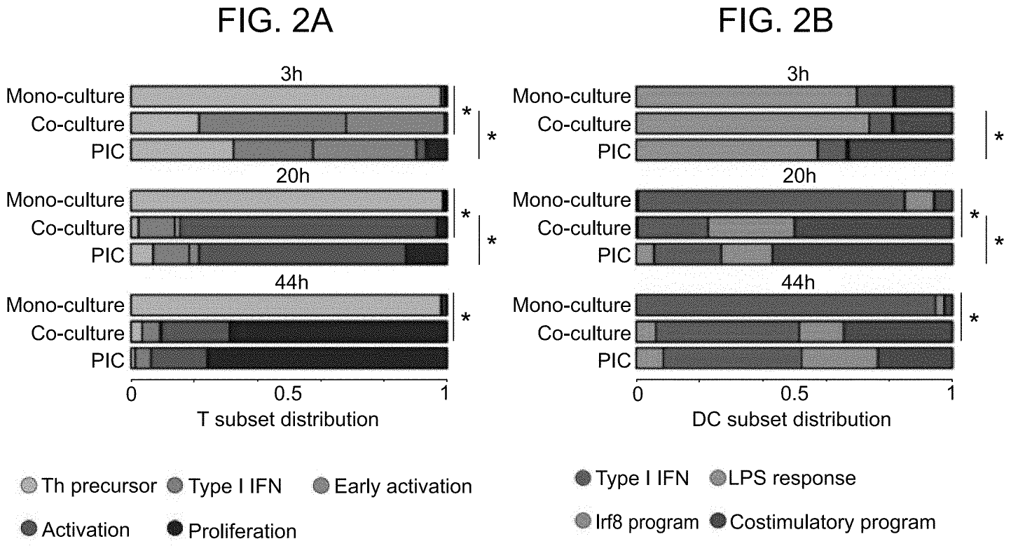

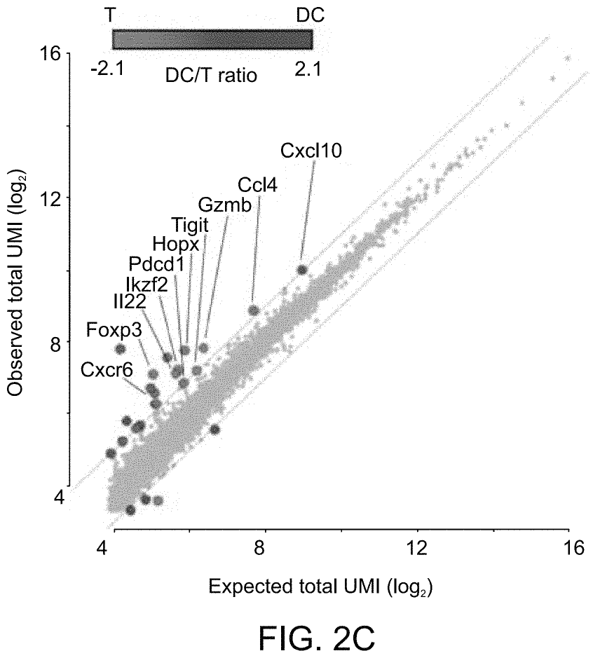

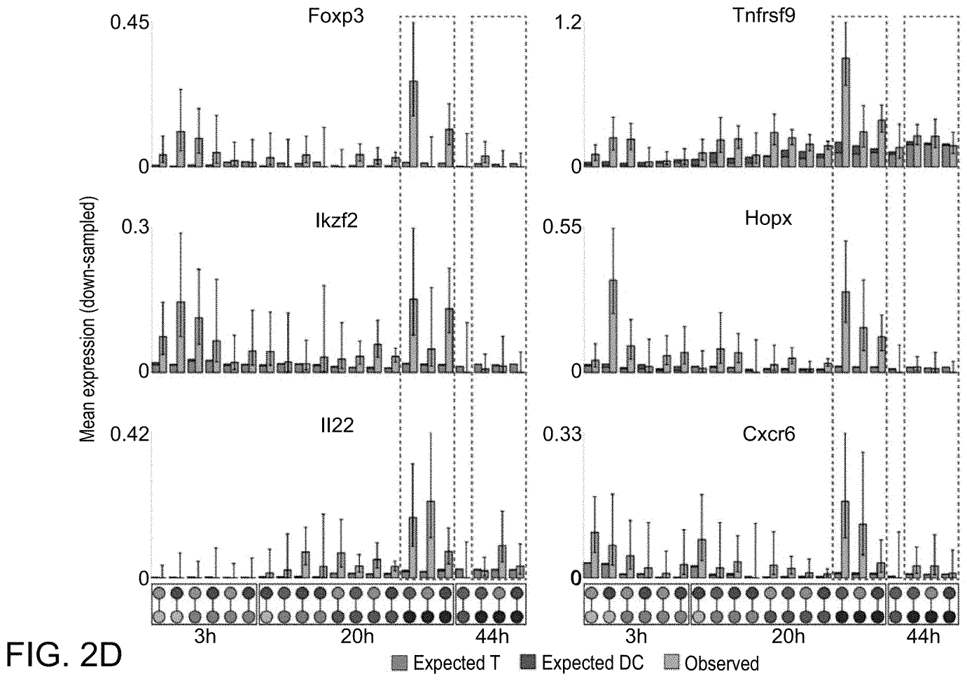

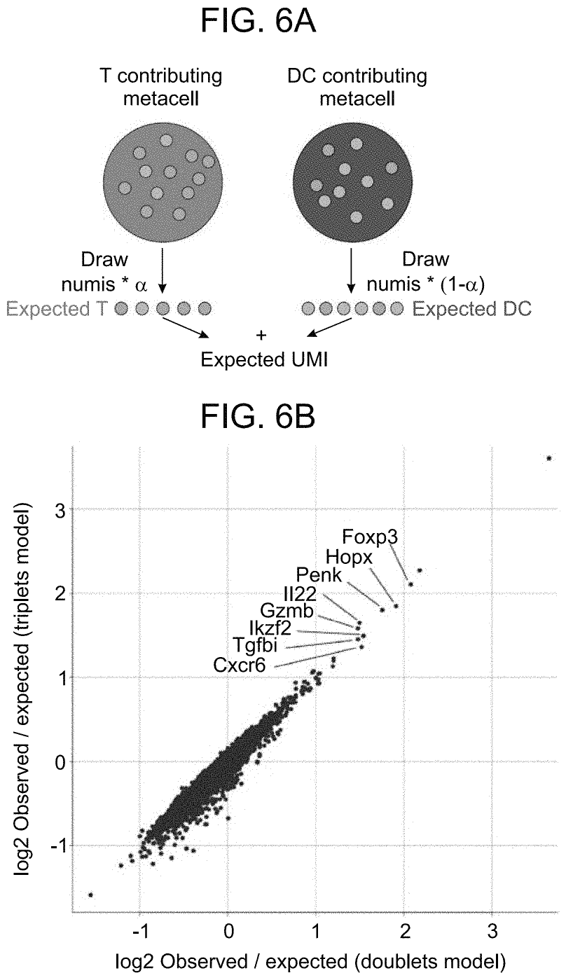

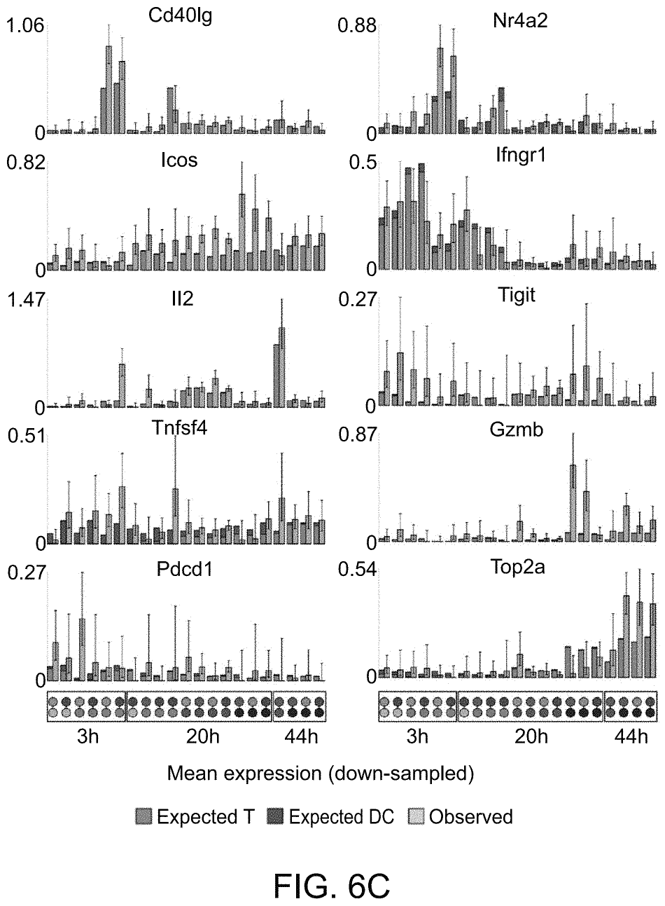

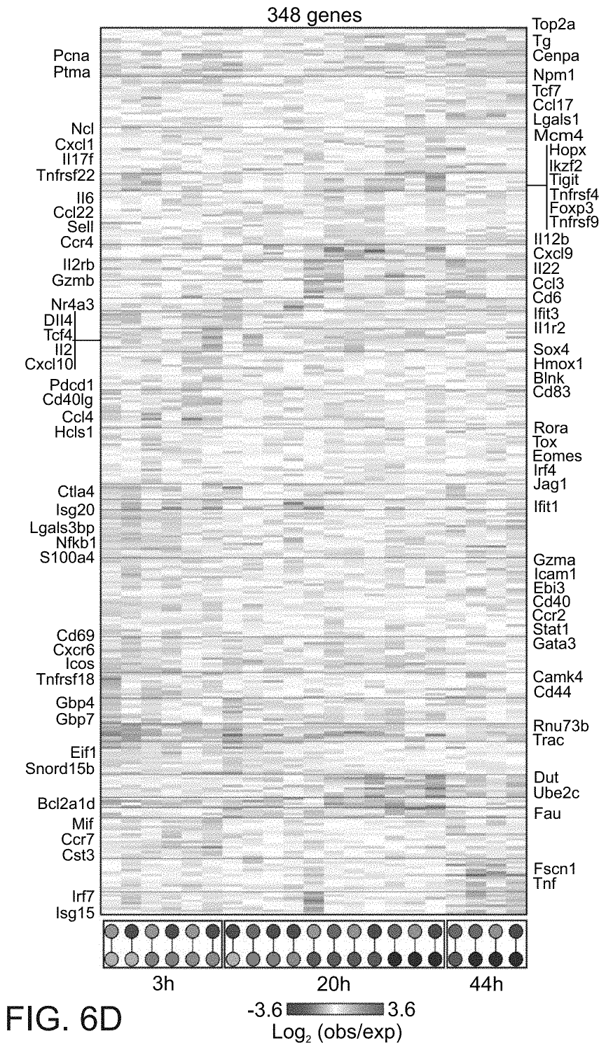

Importantly, the right side of the equation has two parts, one denoting the expected contribution from the T cell, and the other the contribution from the DC (FIGS. 6A-D). In FIG. 2C, the log ratio between these two values was use to color genes as either T or DC-related. We used FDR adjusted χ² test to systematically scan for genes whose observed values diverge from expected in specific groups of PIC (FIGS. 6A-D q<10⁻⁶).

Data availability: RNA-seq data that support the findings of this study was deposited in the Gene Expression Omnibus (GEO) under accession code GSE135382.

Results

Physically Interacting Cells (PIC) can be isolated using carefully calibrated tissue disassociation protocols, fluorescent staining for cell surface epitopes representing distinct cell type markers and Fluorescence Activated Cell Sorting (FACS) (Methods). Sequencing RNA from heterotypic particles using MARS-seq^{16,29} can then provide information about the combined RNA profiles of tens of thousands putatively interacting cells. This newly developed technology (PIC-seq, FIG. 1A), is then analyzed through computational deconvolution of putative complexes and comparative modelling of the distribution of transcriptional states in PIC and single cells. We tested PIC-seq performance on the prototype crosstalk between dendritic cells (DC) and T cells^{30, 31}, utilizing an in vitro model of CD4⁺ T cells isolated from transgenic mice with OVA-specific αβ-TCRs (OT-II) and DC with MHC-II loaded with chicken ovalbumin antigen 323-339 (OVA). We isolated splenic CD11c⁺ DC from C57BL/6 wild type mice and exposed them to OVA in the presence of the TLR4 agonist lipopolysaccharide (LPS). We washed and cultured the LPS-activated and OVA-loaded DC with splenic OT-II CD4 T cells. MARS-seq was performed on 2,387 QC-positive single TCRβ⁺ T cells, and 2,008 QC-positive single CD11c⁺ DC, collected from three time points following co-culture (3, 20 and 44 hours) (FIGS. 1B-E). To assess baseline transcriptional states of non-interacting DC and T cells, we collected additional single cells from mono-cultures of both cell types at matching time points (1,259 QC-positive T cells and 1,848 QC-positive DC). To directly characterize physically interacting cells, we additionally sorted and analyzed 2,726 QC-positive TCRβ⁺CD11c⁺ double positive conjugates (representing the PIC population; FIG. 1B).

PIC-Seq Enriches and Models Interacting Cell Conjugates

FACS analysis of T-DC co-cultured cells revealed a reproducible population (5.68±0.53%) positive for both the beta chain of the T cell receptor (TCRβ) and the DC-specific integrin CD11c (the PIC gate; FIG. 1B). To assess the specificity of true cell-cell interactions formed in culture versus technical and spurious interactions formed during sample processing, we performed two parallel co-cultures, and stained each culture with antibodies against the same epitopes, but associated to different fluorophores (TCRβ-FITC/CD11c-APC and TCRβ-PE/CD11c-APC-Cy7). Following the staining step, we mixed the two cultures and processed them through the PIC-seq sorting pipeline. We observed that spurious particles, combining fluorophores from parallel mixes, are rare, accounting for ˜1% of the PIC gate (FIGS. 5A-H).

To facilitate PIC-seq analysis, we used single cells sorted from single-positive non-conjugated populations (TCRβ⁺ or CD11c⁺) to generate a background MetaCell model for the transcriptional distributions in potentially interacting cells³² (FIG. 1C-E). We assumed sequenced PIC to represent combinations of transcripts (each represented by a unique molecular identifier, UMI) from the derived single cell distributions, superimposing expression of T and DC genes to create a mixed distribution (FIGS. 1F-H; 5A-H). To enable mixed particle deconvolution, we first developed a regression strategy for estimating the fraction of UMIs contributed by the interacting cell types in each conjugate, also denoted the mixing factor (FIG. 1H). Based on the rich cell-type specific information observed in each lineage, simulations demonstrated high accuracy can be derived for estimating the mixing factor (FIGS. 5A-H). Given a background MetaCell model for single cells and a mixing factor per conjugate, we can represent PIC-seq UMIs explicitly as heterotypic mixtures of metacells and infer the maximum likelihood partners contributing to each PIC (FIG. 1G, FIGS. 5A-H; Methods). This defines the basis for downstream analysis of the interaction landscape.

Naturally, the rigid modelling assumptions of this deconvolution strategy are imprecise. First, PIC conjugates may arise from doublets, triplets or larger aggregates. We provide initial estimation of the triplet/doublet composition of any PIC-seq dataset using analysis of synthetic conjugates (doublets or triplets) generated from the single-cell background model (FIGS. 5A-H; Methods). Experimental validations of the composition of particles are also feasible as further discussed below. Second, deconvolution may be biased if the interaction activates de novo transcription, such that it cannot be captured by variation in the background metacells. To bound the effect of this potential bias, we performed cross validation and demonstrate high accuracy in predicting PIC-seq expression of left-out genes based on their deconvoluted single cell components (FIGS. 5A-H). In a similar vein, we observed that differential expression between PIC is highly dependent on the identities of their contributors (FIGS. 5A-H). We note that after observed heterotypic PIC-seq particles are approximated robustly as mixtures of T and DC single cells, we can still identify residual differential expression between empirical PIC-seq conjugates and the single cells inferred as the substrate of the PIC, deriving high resolution view into the regulation of genes in physically interacting cells.

T-DC Interactions Induce T Cell Activation and Differentiation

Following enrichment, sequencing and deconvolution, a PIC-seq experiment can be interrogated to quantify the pairing specificity of interacting cells, in particular when physical interactions are rare and affect only a small fraction of the single cells. We will demonstrate this application below when applying PIC-seq in vivo (FIGS. 3A-L a 4A-I). In addition, when interactions are frequent, PIC-seq can be used to interrogate gene regulatory programs specific to the interaction, after controlling for the single cell transcriptional states of the contributing partners, as we next demonstrate for the OT-II-OVA in vitro model. Co-cultured T cells show strong activation kinetics, with reduction in the relative intensity of the basal T-helper precursor program (Klf2, Sell), early induction of an Interferon type-I response (Stat1, Irf7), and later waves of signaling and metabolic activation (Myc and Npm1) programs, followed by a strong T cell proliferation signature observed after 44 h. Co-cultured DC single cell states exhibit rapid induction of costimulatory molecules (Cd40, Dll4 and Ebi3), with a parallel induction of Irf8 and related genes (Cst3, Ccl22). We validated that these activation programs were dependent on physical interactions by co-culturing T cells and DC for 20 h while maintaining physical separation by using a membrane allowing passage of soluble materials but not cell-cell interactions, and deriving single cell distributions indistinguishable from mono-cultures (633 QC-positive T cells and 897 QC-positive DC). Interestingly, PIC-seq analysis after 3 h and 20 h showed enrichment of interactions in a relatively rare population of proliferating T-cells and a matching enrichment in lymphocyte activating DC (FIGS. 2A-B). As activation and proliferation became prevalent in both T cells and DC after 20 h and 44 h, we focused on identifying differential gene-expression associated with the interaction.

To test for genes expressed specifically in interacting T cells or DC, we compared pooled observed and expected UMIs in PIC conjugates. Expected UMIs were estimated based on inferred PIC mixing factor and T and DC contributing partners (FIG. 2C, FIGS. 6A-D), while also controlling for possible triplet contamination (Methods). We compared each differentially expressed gene to its DC and T-cell expected contributions, using the relative distributions derived from both cell types. Interestingly, PIC-seq identified up-regulation of genes related to T helper differentiation across all PIC particles, including transcription factors (Ikzf2, Foxp3), as well as cytokines, chemokine receptors, and effector genes (Il22, Cxcr6, Pdcd1, Tigit and Tnfrsf9; FIG. 2C). We also identified the chemokine Cxcl10, a chemoattraction and adhesion molecule for T cells, as a DC gene specifically upregulated in PIC (FIG. 2C). Analysis of observed/expected distributions stratified over T and DC identities, showed these same genes were elevated in PIC that were associated with proliferating T-cells after 20 h, and, to a lesser extent, after 44 h when T-cell activation and proliferation had peaked (FIG. 2D, FIGS. 6A-D). We observed less pronounced effect for interaction on DC related genes. Together, the DC-T co-culture model showcases the application of PIC-seq for characterizing the DC-T crosstalk, suggesting preferential DC-T interaction is occurring before massive activation of the T-cell proliferative program. It also demonstrates that heterotypic conjugates induce a proliferation and differentiation program of T helper related genes.

Application of PIC-Seq for Capturing Cellular Interactions in Tissues.

In order to examine whether PIC-seq can be applied to map a broad range of cell-cell interactions in various tissues, we applied PIC-seq on a model of lung development, in which we previously explored intercellular crosstalk, through analysis of ligand-receptor pairs in scRNA-seq data²⁴. Expanding our previous network on ligand-receptor interactions, in which we observed diverse signaling crosstalk between immune and epithelial cells, we focused on physical interactions between immune cells (CD45⁺) and epithelial cells (EPCAM⁺) immediately after birth²⁴. We dissociated neonate lung tissue with the elastase enzyme, which we found preferable for epithelial cell isolation^{24, 33}. Application of PIC-seq on the neonate lung revealed a distinct population (0.15%) positive for both epithelial cells (EPCAM⁺) and immune cells (CD45⁺) surface markers (the PIC gate; FIGS. 7A-J). We combined our previous data with additional 615 QC-positive EPCAM⁺ cells, 441 CD45⁺ cells, and 543 Epcam⁺CD45⁺ PIC, and observed that premature alveolar macrophages (AM), monocytes and DC are over-represented in physical interactions with epithelial cells in neonate lungs, while monocyte-derived macrophages, neutrophils and lymphocytes are depleted (FIGS. 7A-J). We used PIC-seq to define the crosstalk in the epithelial-immune PIC, and identified that premature AM that interacted with alveolar type 1 epithelial cells (AT1) upregulated genes involved in sensing and responding to glucocorticoids (eg. Cxcl2, Sgk1, Chi313, and Fkbp5; FIGS. 7A-J)³⁴⁻³⁶, compared to non-conjugated premature AM.

We next turned back to interactions between T cells and antigen presenting cells (APC), and characterized cellular communications in the draining lymph node (LN). Immune response to infection is initiated through pathogen detection by APC, leading to their migration to the draining LN, where they interact with T cells and initiate an immune cascade¹⁰ (FIG. 3A) with a major impact on infectious disease and cancer^{11, 37}. To characterize T-APC PIC in the draining LN, we first calibrated a suitable tissue dissociation protocol to ensure preservation of interacting conjugates (FIG. 3B). Using these PIC calibrated dissociation conditions, we next applied PIC-seq to characterize the crosstalk of APC and T cells in the LN, following exposure of mice to a classical pathogen immunization model. We performed intradermal injection of fluorescently labeled N. brasiliensis (Nb; helminth), or PBS (control), into the ear pinna of WT mice^{38, 39}. After 48 hours, we extracted and mildly dissociated the auricular draining LN and sorted the cells by their expression of TCRβ and CD11c (FIG. 3C-D). We used FACS analysis to identify a small, yet reproducible, TCRβ⁺CD11c⁺ PIC population (0.31±0.06%), and verified that spurious doublet contribution to the PIC gate was <25% (FIG. 3D). We used confocal microscopy to assess tissue dissociation and sorting efficiency of PIC, and revealed that 87% of the PIC gate events consisting of two or more cells (n=4 biological replicates) showed typical immune synapses associated with a heterotypic doublet structure, while 13% of the particles were likely to represent triplets (mostly two T cells and one DC) (FIG. 3E-F; FIGS. 7A-J). An overall negligible number of conjugates (less than 1/2,000) depicted events representing larger cell clusters. To generate a PIC-seq single-cell background model, we collected 2,610 QC-positive single positive TCRβ⁺ T cells and 2,787 QC-positive CD11c⁺ myeloid cells from 7 Nb infected and 4 PBS control mice, respectively.

As initial analysis identified T regulatory and CD8 memory T cells as enriched within the PIC, we additionally sorted 438 QC-positive ICOS⁺TCRβ⁺, 424 TIGIT⁺TCRβ⁺ and 891 QC-positive CD160⁺TCRβ⁺ T cells, enhancing our cohort with regulatory T cells (Treg) and CD8 memory T cells (FIGS. 7A-J). Metacell analysis of the background single-cell populations revealed high heterogeneity among T cells and APC (FIGS. 3G-I). We grouped T metacells by their transcription profiles into naïve T cells (Tcf7), naïve CD8⁺ (Cd8a, Cd8b1), CD8 memory (Ccl5, Ly6c2, Ctla2a and Il2rb), activated T (cell cycle genes, Srm and Npm1), and Tregs (Ctla4, Icos, Foxp3, Il2ra). APC were similarly divided into migratory DC (Fscn1, Ccl22), cDC1 (Xcr1, Clec9a), cDC2 (Cd209a, Sirpa, Itgam)⁴⁰, monocytes (Lyz2), and pDC (Siglech, Tcf4). As reported previously, Nb-injected LN differed from PBS controls by an increase in the monocyte population³⁸ (FIGS. 7A-J). Moreover, APC that specifically presented Nb antigens (466 Ag⁺CD11c⁺ cells) were mainly associated with the migratory DC and monocyte subsets (FIGS. 7A-J).

We next sorted and sequenced 1,681 QC-positive TCRβ⁺CD11c₊ PIC from Nb-injected mice and 986 PIC from PBS controls. Using the PIC-seq pipeline we inferred for each PIC the putative identities of its T and APC partners and their mixing factor (FIGS. 3J-L). Consistently with the microscopy data, conjugate simulations estimated frequency of 4% for triplets involving two T-cells and 3% for triplets involving two DCs (FIGS. 7A-J). This allowed us to filter high probability triplets from the PIC dataset (FIGS. 7A-J). Similar to the co-culture in vitro experiment, we observed that differential expression between PIC is highly dependent on the identities of their contributing T and APC.

Regulatory T Cells Frequently Interact with Myeloid Cells in the Lymph Node

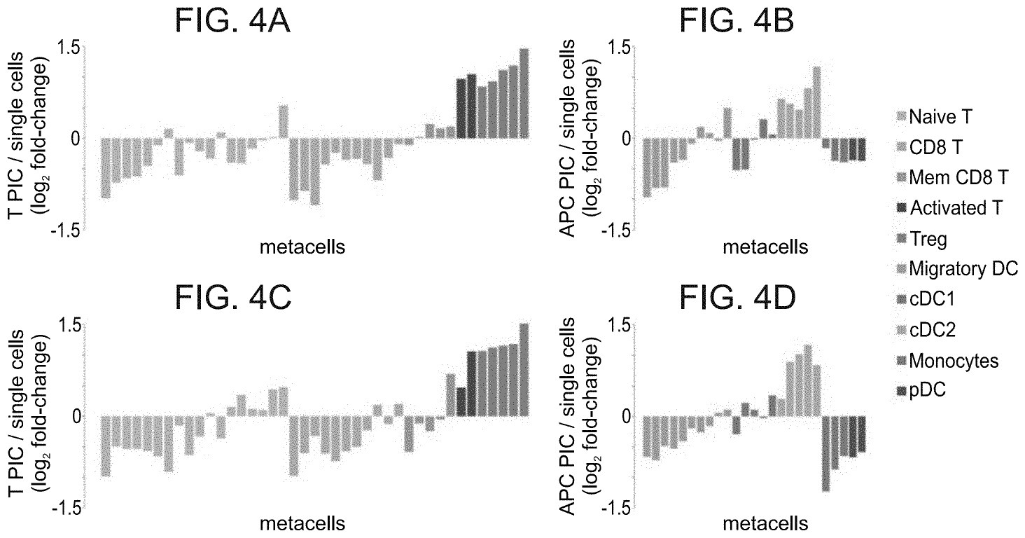

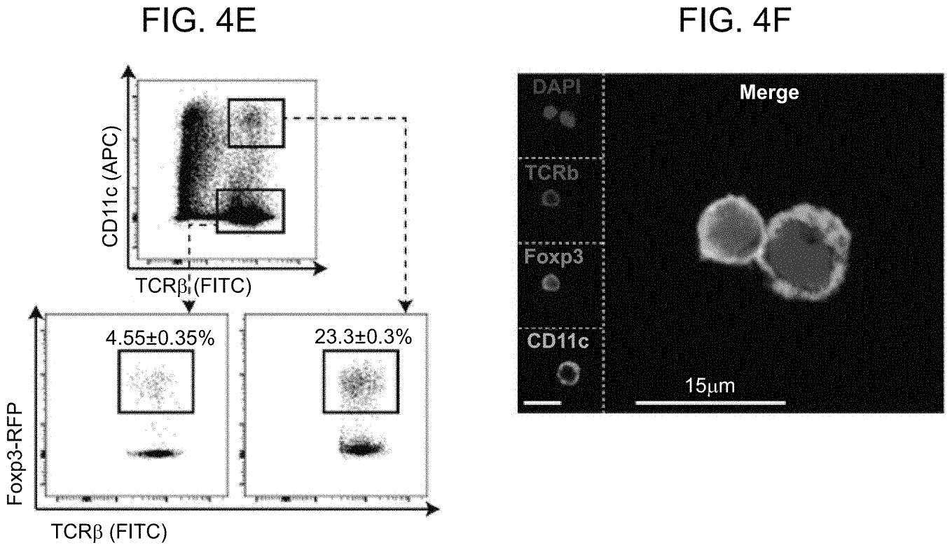

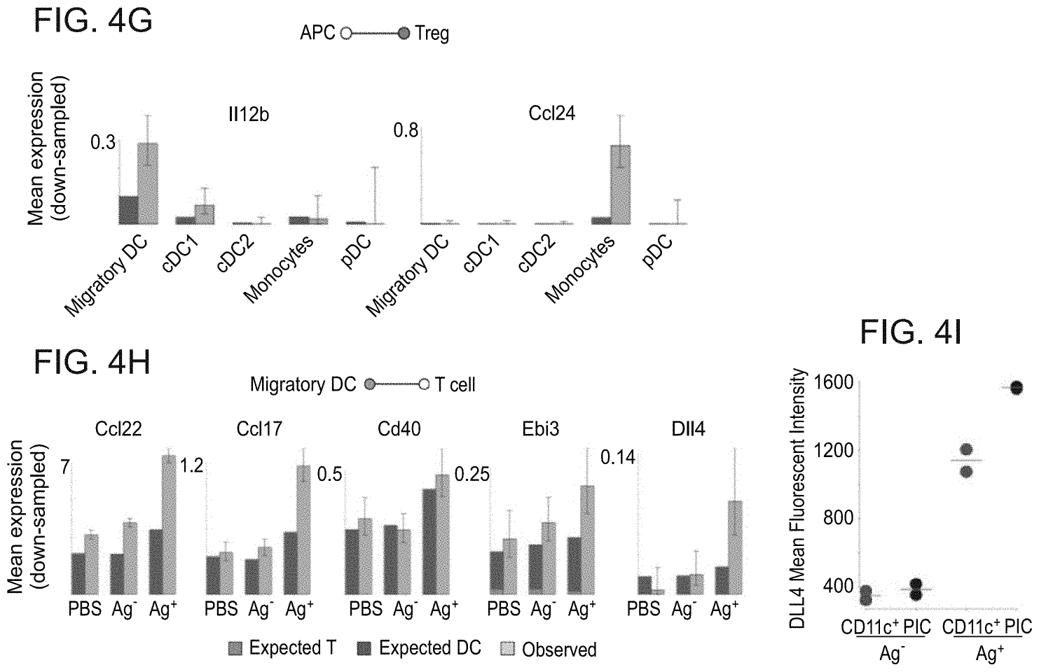

We systematically assessed the interaction capacities and preferences of all T and myeloid subsets. Analysis of the control samples (PBS), unexpectedly showed that PIC-derived T cells were highly enriched for the Treg (4.3 fold; p<10⁻¹⁰ by a two-tailed Fisher's exact test) and activated T subsets (6 fold; p<10⁻¹⁰), combining to more than 37% of all T-myeloid PIC, and depleted for the naïve and CD8 naïve T subsets (p<10⁻¹⁰; FIG. 4A). Myeloid contribution to PIC was enriched for the cDC2 (2 fold) and depleted for the migratory DC and pDC subsets (p<10⁻¹⁰; FIG. 4B). We observed similar interaction specificities in Nb-injected samples (FIGS. 4C-D; 5.5 fold enrichment in Treg and 3.5 fold enrichment in activated CD8 T cells), despite the overall change in relative frequencies of the myeloid (and in particular monocyte) populations.

These results were reproducible across biological replicates. Together, PIC-seq analysis of myeloid-T cell interactions identifies a high frequency of interactions between Treg and myeloid cells in the LN, independently of infection status.

In order to validate the high capacity of Treg to interact with APC in the naïve auricular LN, we quantified Treg frequencies in PIC and single cells using a transgenic mouse expressing a fluorescent Foxp3 reporter (Foxp3-RFP mice) by FACS and confocal microscopy. Further supporting PIC-seq results, while the Foxp3+ Treg population represented only a small fraction (4.55%) of the TCRβ⁺ single positive population, Foxp3⁺ Tregs constituted 23.3% of the TCRβ⁺CD11c⁺ PIC population (FIG. 4E). Sorting Foxp3⁺ PIC conjugates, followed by their fixation and imaging, verified that these PIC represented pairs of CD11c⁺ APC, conjugated to Foxp3-expressing Treg (FIG. 4F). Comparing the observed transcription of Treg-APC PIC to their expected values according to PIC-seq, discovered PIC-specific gene expression. Conjugates of Treg and migratory DC specifically upregulated Interleukin 12 p40 subunit (Mb), an important stimulatory T cell cytokine, while Treg-monocyte PIC upregulated Ccl24 (FIG. 4G). This suggests that interactions of Treg with different myeloid subsets is mediated by specific signaling pathways.

At early time points post-infection, the pathogen specific crosstalk of innate-adaptive cells may be limited to small subsets of APC and antigen-specific T cells, an effect which might be overlooked even with state-of-the-art technologies because of the dominance of background bystander cells (FIG. 3A). To focus on rare Nb-specific interactions, we next performed PIC-seq on 309 QC-positive Ag⁺TCRβ₊CD11c⁺ PIC (capturing less than 2×10⁻⁵ of total events), representing complexes of T cells with APC that presented Nb-derived antigens. We observed that PIC of Nb-presenting migratory DC specifically upregulated a gene module consisting of the chemo-attractants Ccl22 and Ccl17, as well as the costimulatory genes Cd40, Ebi3, and Dll4 (FIG. 4H), compared to their expected UMI based on the background model. This trend was specific to the Ag⁺ PIC, and was reduced in Ag⁻ or PBS-injected LN derived PIC, suggesting it is part of an antigen-specific PIC-exclusive response, which might prime the pathogen-induced T cell response. Importantly, the same gene module was induced in the in vitro PIC derived from co-cultures of OVA-loaded DC and OTII-CD4⁺ T cells. In line with these data, Cd40, Ebi3 and Dll4 were previously shown to play a role in maturation of the adaptive immune response in the form of CD4⁺ T helper and cytotoxic T cells⁴¹⁻⁴⁴. We subsequently validated that Ag⁺ PIC expressed higher levels of the protein DLL4 compared to singlet Ag⁺ DC and Ag⁻ PIC and non-conjugated DC (FIG. 41). Taken together, we highlighted a unique molecular module of increased costimulatory behavior by Nb-presenting migratory DC that are physically interacting with T cells. Thus, PIC-seq represents a unique technology that enables in vivo characterization and molecular screening of cell-cell interactions, with high potential to identify novel targets of cell-cell signaling molecules.

### REFERENCES

1. Banchereau, J. & Steinman, R. M. Dendritic cells and the control of immunity. Nature 392, 245-252 (1998).
2. Freeman, S. A. & Grinstein, S. Phagocytosis: receptors, signal integration, and the cytoskeleton. Immunol Rev 262, 193-215 (2014).
3. Lanier, L. L. Natural killer cell receptor signaling. Curr Opin Immunol 15, 308-314 (2003).
4. Jaitin, D. A. et al. Lipid-Associated Macrophages Control Metabolic Homeostasis in a Trem2-Dependent Manner. Cell 178, 686-698 e614 (2019).
5. Li, H. et al. Dysfunctional CD8 T Cells Form a Proliferative, Dynamically Regulated Compartment within Human Melanoma. Cell 176, 775-789 e718 (2019).
6. Ledergor, G. et al. Single cell dissection of plasma cell heterogeneity in symptomatic and asymptomatic myeloma. Nat Med 24, 1867-1876 (2018).
7. Gerber, T. et al. Single-cell analysis uncovers convergence of cell identities during axolotl limb regeneration. Science 362 (2018).
8. Keren-Shaul, H. et al. A Unique Microglia Type Associated with Restricting Development of Alzheimer's Disease. Cell 169, 1276-1290 e1217 (2017).
9. Steinman, R. M. & Banchereau, J. Taking dendritic cells into medicine. Nature 449, 419-426 (2007).
10. Zhu, J., Yamane, H. & Paul, W. E. Differentiation of effector CD4 T cell populations (*). Annu Rev Immunol 28, 445-489 (2010).
11. Binnewies, M. et al. Unleashing Type-2 Dendritic Cells to Drive Protective Antitumor CD4(+) T Cell Immunity. Cell 177, 556-571 e516 (2019).
12. van Panhuys, N. Studying Dendritic Cell-T Cell Interactions Under In Vivo Conditions. Methods Mol Biol 1584, 569-583 (2017).
13. Barnden, M. J., Allison, J., Heath, W. R. & Carbone, F. R. Defective TCR expression in transgenic mice constructed using cDNA-based alpha- and beta-chain genes under the control of heterologous regulatory elements. Immunol Cell Biol 76, 34-40 (1998).
14. Macosko, E. Z. et al. Highly Parallel Genome-wide Expression Profiling of Individual Cells Using Nanoliter Droplets. Cell 161, 1202-1214 (2015).
15. Klein, A. M. et al. Droplet barcoding for single-cell transcriptomics applied to embryonic stem cells. Cell 161, 1187-1201 (2015).
16. Jaitin, D. A. et al. Massively parallel single-cell RNA-seq for marker-free decomposition of tissues into cell types. Science 343, 776-779 (2014).
17. Rodrigues, S. G. et al. Slide-seq: A scalable technology for measuring genome-wide expression at high spatial resolution. Science 363, 1463-1467 (2019).
18. Eng, C. L. et al. Transcriptome-scale super-resolved imaging in tissues by RNA seqFISH. Nature 568, 235-239 (2019).
19. Keren, L. et al. A Structured Tumor-Immune Microenvironment in Triple Negative Breast Cancer Revealed by Multiplexed Ion Beam Imaging. Cell 174, 1373-1387 e1319 (2018).
20. Wang, X. et al. Three-dimensional intact-tissue sequencing of single-cell transcriptional states. Science 361 (2018).
21. Medaglia, C. et al. Spatial reconstruction of immune niches by combining photoactivatable reporters and scRNA-seq. Science 358, 1622-1626 (2017).
22. Chen, K. H., Boettiger, A. N., Moffitt, J. R., Wang, S. & Zhuang, X. RNA imaging. Spatially resolved, highly multiplexed RNA profiling in single cells. Science 348, aaa6090 (2015).
23. Vento-Tormo, R. et al. Single-cell reconstruction of the early maternal-fetal interface in humans. Nature 563, 347-353 (2018).
24. Cohen, M. et al. Lung Single-Cell Signaling Interaction Map Reveals Basophil Role in Macrophage Imprinting. Cell 175, 1031-1044 e1018 (2018).
25. Camp, J. G. et al. Multilineage communication regulates human liver bud development from pluripotency. Nature 546, 533-538 (2017).
26. Szczerba, B. M. et al. Neutrophils escort circulating tumour cells to enable cell cycle progression. Nature 566, 553-557 (2019).
27. Halpern, K. B. et al. Paired-cell sequencing enables spatial gene expression mapping of liver endothelial cells. Nat Biotechnol 36, 962-970 (2018).
28. Bois set, J. C. et al. Mapping the physical network of cellular interactions. Nat Methods 15, 547-553 (2018).
29. Keren-Shaul, H. et al. MARS-seq2.0: an experimental and analytical pipeline for indexed sorting combined with single-cell RNA sequencing. Nat Protoc 14, 1841-1862 (2019).
30. Celli, S., Lemaitre, F. & Bousso, P. Real-time manipulation of T cell-dendritic cell interactions in vivo reveals the importance of prolonged contacts for CD4+ T cell activation. Immunity 27, 625-634 (2007).
31. Kane, L. P., Lin, J. & Weiss, A. Signal transduction by the TCR for antigen. Curr Opin Immunol 12, 242-249 (2000).
32. Baran, Y. et al. MetaCell: analysis of single-cell RNA-seq data using K-nn graph partitions. Genome Biol 20, 206 (2019).
33. Treutlein, B. et al. Reconstructing lineage hierarchies of the distal lung epithelium using single-cell RNA-seq. Nature 509, 371-375 (2014).
34. Bird, A. D. et al. Identification of glucocorticoid-regulated genes that control cell proliferation during murine respiratory development. J Physiol 585, 187-201 (2007).
35. Itani, 0.A., Liu, K. Z., Cornish, K. L., Campbell, J. R. & Thomas, C. P. Glucocorticoids stimulate human sgk1 gene expression by activation of a GRE in its 5′-flanking region. Am J Physiol Endocrinol Metab 283, E971-979 (2002).
36. Binder, E. B. The role of FKBPS, a co-chaperone of the glucocorticoid receptor in the pathogenesis and therapy of affective and anxiety disorders. Psychoneuroendocrinology 34 Suppl 1, S186-195 (2009).
37. Bonifaz, L. C. et al. In vivo targeting of antigens to maturing dendritic cells via the DEC-205 receptor improves T cell vaccination. J Exp Med 199, 815-824 (2004).
38. Blecher-Gonen, R. et al. Single-Cell Analysis of Diverse Pathogen Responses Defines a Molecular Roadmap for Generating Antigen-Specific Immunity. Cell Syst 8, 109-121 e106 (2019).
39. Connor, L. M., Tang, S. C., Camberis, M., Le Gros, G. & Ronchese, F. Helminth-conditioned dendritic cells prime CD4+ T cells to IL-4 production in vivo. J Immunol 193, 2709-2717 (2014).
40. Merad, M., Sathe, P., Helft, J., Miller, J. & Mortha, A. The dendritic cell lineage: ontogeny and function of dendritic cells and their subsets in the steady state and the inflamed setting. Annu Rev Immunol 31, 563-604 (2013).
41. Fujii, S., Liu, K., Smith, C., Bonito, A. J. & Steinman, R. M. The linkage of innate to adaptive immunity via maturing dendritic cells in vivo requires CD40 ligation in addition to antigen presentation and CD80/86 costimulation. J Exp Med 199, 1607-1618 (2004).
42. Bennett, S. R. et al. Help for cytotoxic-T-cell responses is mediated by CD40 signalling. Nature 393, 478-480 (1998).
43. Ohshima, Y. et al. OX40 costimulation enhances interleukin-4 (IL-4) expression at priming and promotes the differentiation of naive human CD4(+) T cells into high IL-4-producing effectors. Blood 92, 3338-3345 (1998).
44. Laky, K., Evans, S., Perez-Diez, A. & Fowlkes, B. J. Notch signaling regulates antigen sensitivity of naive CD4+ T cells by tuning co-stimulation. Immunity 42, 80-94 (2015).
45. Tabula Muris, C. et al. Single-cell transcriptomics of 20 mouse organs creates a Tabula Muris. Nature 562, 367-372 (2018).
46. Zeisel, A. et al. Molecular Architecture of the Mouse Nervous System. Cell 174, 999-1014 e1022 (2018).
47. Regev, A. et al. The Human Cell Atlas. Elife 6 (2017).
48. Kiselev, V. Y., Andrews, T. S. & Hemberg, M. Challenges in unsupervised clustering of single-cell RNA-seq data. Nat Rev Genet 20, 273-282 (2019).
49. Wolock, S. L., Lopez, R. & Klein, A. M. Scrublet: Computational Identification of Cell Doublets in Single-Cell Transcriptomic Data. Cell Syst 8, 281-291 e289 (2019).
50. McGinnis, C. S., Murrow, L. M. & Gartner, Z. J. DoubletFinder: Doublet Detection in Single-Cell RNA Sequencing Data Using Artificial Nearest Neighbors. Cell Syst 8, 329-337 e324 (2019).
51. DePasquale, E. A. et al. DoubletDecon: cell-state aware removal of single-cell RNA-seq doublets. BioRxiv, 364810 (2018).
52. Krishnaswamy, S. et al. Systems biology. Conditional density-based analysis of T cell signaling in single-cell data. Science 346, 1250689 (2014).
53. Pasqual, G. et al. Monitoring T cell-dendritic cell interactions in vivo by intercellular enzymatic labelling. Nature 553, 496-500 (2018).
54. Akkaya, B. et al. Regulatory T cells mediate specific suppression by depleting peptide-MHC class II from dendritic cells. Nat Immunol 20, 218-231 (2019).
55. Yan, J., Liu, B., Shi, Y. & Qi, H. Class II MHC-independent suppressive adhesion of dendritic cells by regulatory T cells in vivo. J Exp Med 214, 319-326 (2017).
56. Sakaguchi, S., Yamaguchi, T., Nomura, T. & Ono, M. Regulatory T cells and immune tolerance. Cell 133, 775-787 (2008).
57. Li, D. et al. VCAM-1(+) macrophages guide the homing of HSPCs to a vascular niche. Nature 564, 119-124 (2018).
58. Scott, L. M., Priestley, G. V. & Papayannopoulou, T. Deletion of alpha4 integrins from adult hematopoietic cells reveals roles in homeostasis, regeneration, and homing. Mol Cell Biol 23, 9349-9360 (2003).
59. Deczkowska, A. et al. Disease-Associated Microglia: A Universal Immune Sensor of Neurodegeneration. Cell 173, 1073-1081 (2018).
60. Camberis, M. et al. Evaluating the in vivo Th2 priming potential among common allergens. J Immunol Methods 394, 62-72 (2013).
61. Paul, F. et al. Transcriptional Heterogeneity and Lineage Commitment in Myeloid Progenitors. Cell 163, 1663-1677 (2015).

### Example 2

PIC-Seq Application on Tumor and Adjacent Normal Tissues Derived from Stage-I Biopsies of NSCLC Patients

Methods

Tissue dissociation: To achieve single-cell suspension in the human NSCLC specimens, 0.1-0.4 gr of tumor and adjacent normal tissues were cut into small pieces and then mechanically dissociated by pipetting. Tissues were suspended with CO₂ Independent Medium (Thermo Fisher SCIENTIFIC) supplemented with DNase (100 m/ml, Sigma-Adrich) and collagenase IV (0.5 mg/ml, Worthington), and incubated at 37° C. for 20 min, with frequent agitation.

To achieve single-cell suspensions in the murine model, tumors were cut into small pieces, and suspended with RPMI-1640 supplemented with DNase (28 μg/ml, Sigma-Adrich) and collagenase IV (1 mg/ml, Worthington). Tissues were homogenized by GentleMacs tissue homogenizer (Miltenyi Biotec), and incubated at 37° C. for 10 min, with frequent agitation. This tissue dissociation procedure was performed twice, for each tumor.

To achieve single-cell suspensions from tdLN and cLN, tissues were digested in Isocoves modified Dulbeccos medium (IMDM; Sigma-Aldrich) medium. For mild dissociation, tissues were supplemented with Liberase-TL (100 μg/ml, Roche) and DNase I (100 μg/ml, Roche), and incubated with frequent agitation at 37° C. for 20 min.

After all dissociation procedures, cells were washed with cold PBS, filtered through a 100 μm cell strainer, and centrifuged at 380 g for 5 min at 4° C.

Results

In order to characterize and molecularly dissect physical interactions between myeloid and T cells in the tumor microenvironment (TME), we applied the PIC-seq technology, on clinical samples of early human non-small cell lung carcinoma (NSCLC) lesions. We profiled treatment-naïve surgical resections obtained from eight NSCLC patients, and compared tumor-involved to adjacent tumor-free tissues. We optimized lung tissue dissociation to preserve physiological cell conjugates, and sorted single CD64⁺CD11c⁺ myeloid cells and TCRβ⁺ T cells, as well as CD64⁺CD11c⁺TCRβ⁺ conjugates of physically interacting cells (PICs; FIGS. 1A-B, Methods). Overall, we sequenced 3,688 Quality Control (QC)-positive single TCRβ⁺ T cells and 4,529 QC-positive single CD11c⁺CD64⁺ myeloid cells harvested from tumor and adjacent normal tissues, and used the Metacell package to create a background model of single cell states in the TME and normal tissues (FIGS. 1C-D). We controlled for cross-patient batch effect by confirming that each metacell groups cells from multiple patients. We grouped T metacells, according to hallmark gene expression, into naïve T (TCF7, IL7R), CD8⁺ T (CD8A, CD8B), cytotoxic T lymphocytes (CTLs; GNLY, GZMB, PRF1), dysfunctional (exhausted) CD8⁺ T (DysCD8; GZMK, LAG3, HAVCR2), CD4⁺PD-1⁺CXCL13⁺ T (CD4, CXCL13, PDCD1, IL21), and regulatory CD4⁺ T (Treg; FOXP3, IL2RA) cells. We similarly grouped myeloid cells into two subsets of monocytes, based on the high expression levels of the VCAN and CD31 (PECAM1) genes, respectively, and into four macrophage subsets: Tumor associated macrophage (TAM) subsets I and II (TREM2 and MMP9, respectively); regulatory macrophages (Mreg-Mac; GPNMB, TREM2 and APOE) and Type I interferon genes expressing macrophages (Mac Type I IFN; CXCL10 and IFI30). Additional myeloid subsets included three groups of DCs: classical DC type I (cDC1; XCR1, CLEC9A), classical DC type II (cDC2; CLEC10A, CD1C, BHLHE40), and mature DC enriched in immunoregulatory molecules (mregDC; FSCN1, CCL22, CCL19). We found that the CD11c⁺CD64⁺ populations in some of the patients were enriched in natural killer cells (low in TRBC2 and CD8A, high in TRDC and KLRC1), which we grouped into two subsets (NK I; NCAM1, XCL1 and NK II; CX3CR1). For both the T and myeloid compartments, we identified consistent differences in cell composition between tumor and adjacent tumor free tissues (FIG. 8D). Correlation analysis further supported the restriction of different cell types to either tumor or normal tissues (FIG. 8E). Specifically, Mreg-Mac, TAM I, TAM II, mregDC and cDC2 from the myeloid compartment, together with Treg, CD4⁺PD-1⁺CXCL13⁺ T and DysCD8 T cells were enriched in the TME, while the monocytes subsets, naïve T cells, and CTLs were enriched in the adjacent normal tissues consistently across sampled patients (FIG. 8F). Importantly, these results are in line with a previous NSCLC maps, and therefore can serve as a baseline model to investigate the molecular programs defining the major cellular interactions in the TME.

We identified QC-positive CD64₊CD11c₊TCRβ₊ PICs with heterotypic gene expression and removed putative T-NK PICs due to low specificity of their PIC-seq deconvolution. We overall modeled 591 QC-positive PICs by inference of their T cell and myeloid cell identities as described in the methods.

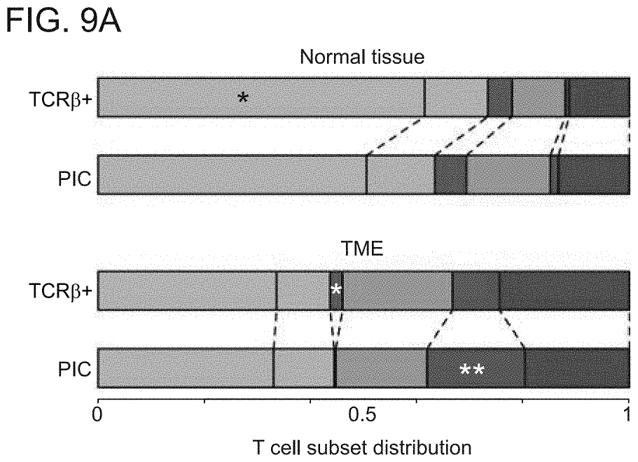

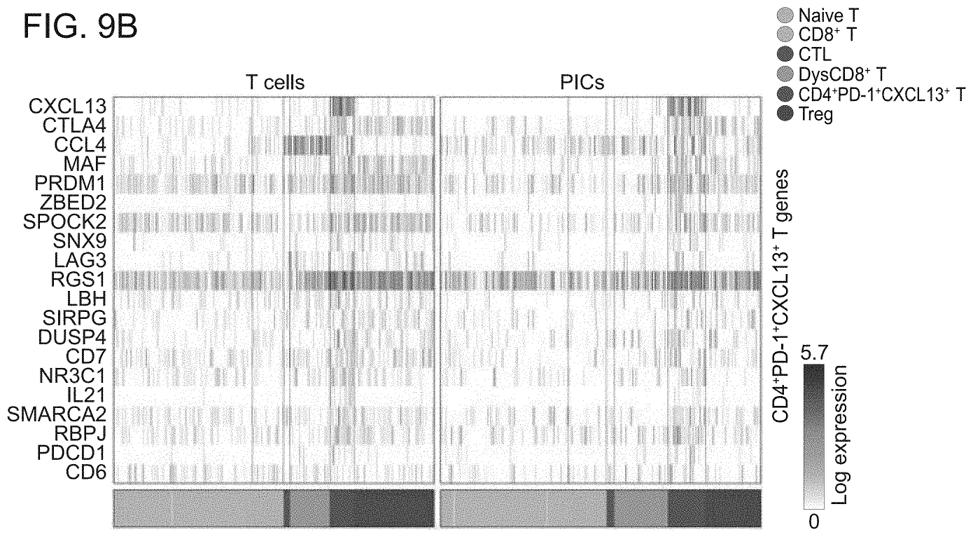

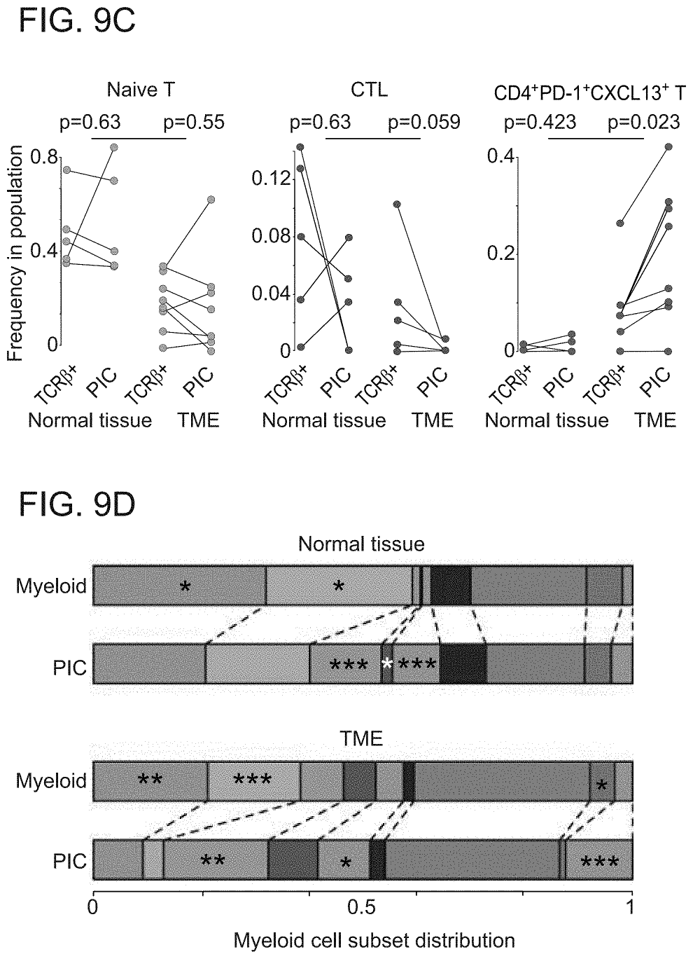

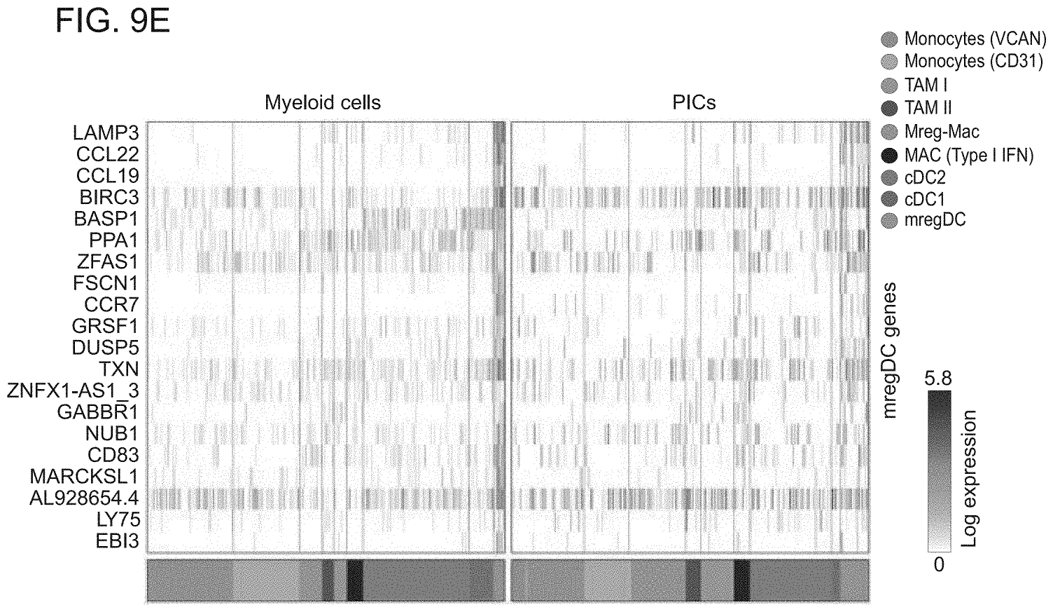

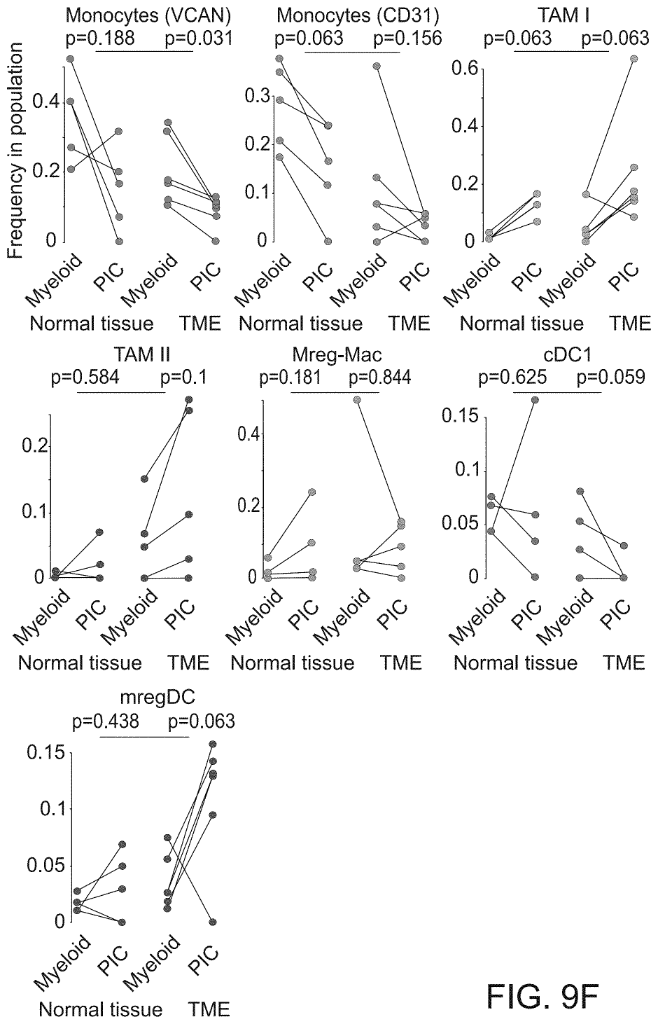

The PIC-seq pipeline utilizes a detailed background model of the singlet populations contributing to the PIC conjugates to facilitate the estimation of interaction preferences, and assign for each PIC the most probable pair of contributing singlet identities (FIGS. 9A-F). In normal lung tissues, we did not observe specific pairings of T subsets, with a slight reduction of naïve T cells in PICs (P=0.025, FDR adjusted two tailed Fisher's exact test FIG. 9A). In contrast, and to our surprise, tumor PICs showed a significant reduction of CTL (P=0.0044), alongside a significant enrichment of CD4⁺PD-1⁺CXCL13⁺ T cells (P=1.22×10⁻⁵) (FIG. 9A). The identification of interactive CD4⁺PD-1⁺CXCL13⁺ T cells within PICs was based on specific genes including CXCL13, MAF, ZBED2, PRDM1, and SNX9 (FIG. 9B). Importantly, these interaction preferences were consistent across all but one profiled patient (FIG. 9C). Myeloid cell contribution to T-Myeloid PIC was characterized by a reduction in both monocyte subsets, and enrichment of TAM-I and Mreg-Mac subsets. This was observed in both normal (P=0.003, 0.04, 5.4×10⁻¹² and 6×10⁻⁶, respectively) and tumor tissues (P=0.0001, 1.2×10⁻⁶, 1.7×10⁻⁵ and 0.04; FIG. 9D). Importantly, we also observed a significant enrichment in mregDC frequency (P=6.04×10⁻⁶), alongside a depletion in cDC1 (P=0.046) specifically in TME PICs (FIG. 9D). mregDC annotation in PICs was based on the expression of unique gene transcripts identified in singlets (e.g. LAMP3, CCL22, CCL19, BIRC3, FSCN1, CCR7; FIG. 9E). These results were consistent across all but one profiled patient (FIG. 9F). Taken together, by investigating the cellular interaction repertoire represented by PICs, we identified distinct cell states, namely CD4⁺PD-1⁺CXCL13⁺ T cells and mregDC, exhibiting high interaction capacities specifically in the TME, but not in the normal lung tissue.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of determining cell members of a cell cluster in a tissue of interest comprising:
(a) receiving a transcriptome of the cell cluster;
(b) accessing a computer readable medium storing a library having a plurality of entries, each entry having a predicted transcriptome of a cell cluster and a set of identities of known cell types forming said cell cluster;
(c) searching said library for an entry having a predicted transcriptome matching said received transcriptome; and
(d) extracting from said entry a corresponding set of identities, thereby determining the cell members of the cell cluster in a tissue, the cell members being said corresponding set of identities, wherein the cell members physically interact with one another in the cell cluster.

2. The method of claim 1 wherein said cell members physically interact via a receptor ligand interaction in the cell cluster.

3. The method of claim 1, wherein said known cell types comprises at least 5 different cell types.

4. The method of claim 1, wherein each of said cell types comprises a unique surface marker or a unique combination of cell surface markers.

5. The method of claim 1, wherein said tissue is not hepatic tissue.

6. The method of claim 1, wherein said cell cluster consists of two or three cells.

7. A computer software product, comprising a computer-readable medium in which program instructions are stored, which instructions, when read by a data processor, cause the data processor to receive a transcriptome of a cell cluster, and to execute the method according to claim 1.

8. An apparatus for determining cell members of a cell cluster in a tissue of interest, the apparatus comprising a data processor configured for receiving a transcriptome of a cell cluster, and for executing the method according to claim 1.

9. A method of identifying cell members of a cell cluster in a tissue of interest:
(a) isolating a plurality of single cells of different cell types from the tissue of interest on the basis of expression of a unique cell marker;
(b) determining the transcriptomes of said single cells of different cell types;
(c) obtaining predictions of the transcriptomes of a plurality of clusters of non-identical cells of said plurality of single cells from said transcriptomes of said single cells, each of said plurality of clusters comprising a unique combination of cells, wherein the predictions are based on the null hypothesis that the transcriptome of a cluster is the sum of a transcriptome of the individual cells of the cluster when in a single cell state;
(d) isolating a cluster of cells from said tissue;
(e) performing transcriptome analysis of said cluster of cells so as to obtain the true transcriptome of said cluster;
(f) comparing said predictions to said true transcriptome; and
(g) identifying the members of the cell cluster by selecting the transcriptome prediction that has the closest identity to said true transcriptome.

10. The method of claim 9, wherein said different cell types comprises at least 5 different cell types.

11. The method of claim 9, wherein said cluster consists of two or three cells.

12. The method of claim 9, further comprising identifying genes of the cells whose transcription is regulated by cell clustering following step (g).

13. The method of claim 9, further comprising determining the abundance of a cell cluster of a particular combination.

14. A method of ascertaining a status of a tissue comprising identifying cell members of cell clusters of the tissue according to the method of claim 9, wherein a presence or an increase in a number of cell clusters comprising a combination of cell members indicative of a tissue status, is indicative of a status of the tissue.

15. The method of claim 14, wherein said status of a tissue comprises a disease.

16. The method of claim 15, wherein the disease is selected from the group consisting of cancer, an infectious disease, fibrosis and an immune disease.

17. A method of identifying a gene whose transcription is regulated by cell clustering comprising:
(a) obtaining data of the transcriptome of a first cell of the cluster, wherein said transcriptome is obtained when said first cell is in a single-cell state;
(b) obtaining data of the transcriptome of a second cell of the cluster, wherein said transcriptome is obtained when said second cell is in a single-cell state, wherein said first cell and said second cell express non-identical cell surface markers;
(c) obtaining a prediction of the transcriptome of a cluster of said first cell and said second cell from said data of the transcriptome of said first cell in a single-cell state and said data of the transcriptome of said second cell in a single cell state, using the null hypothesis that said transcriptome of said cluster is the combination of said transcriptome of said first cell in said single cell state and said transcriptome of said second cell in said single cell state;
(d) isolating a cluster of cells which comprise said first cell and said second cell from a heterogeneous population of cells;
(e) performing transcriptome analysis of said cluster of cells so as to obtain the true transcriptome of said cluster; and
(f) comparing said prediction to said true transcriptome, wherein expression of a gene of said true transcriptome that is statistically significantly altered is indicative of a gene that is regulated by cell clustering.

18. The method of claim 17, wherein said heterogeneous population of cells comprises at least 5 cell types.

19. The method of claim 18, wherein said cluster consists of two cells or three cells.

20. The method of claim 18, further comprising isolating said first cell and said second cell from said heterogeneous population of cells such that they are in a single cell state prior to step (a).
